(19) United States
(12) Patent Application Publication
(10) Pub. No.: US 2024/0366295 A1
(43) Pub. Date: Nov. 7, 2024

(54) Tissue Ablation and Lesion Assessment System

(71) Applicant: SIGT, LLC, Columbia, MD (US)

(72) Inventor: Parag Karmarkar, Columbia, MD (US)

(73) Assignee: SIGT, LLC, Columbia, MD (US)

(21) Appl. No.: 18/292,274

(22) PCT Filed: Jul. 26, 2022
(86) PCT No.: PCT/US22/38386

§ 371 Date: Jan. 25, 2024

Related U.S. Application Data
(60) Provisional application No. 63/225,937, filed on Jul. 26, 2021.
(60) Provisional application No. 63/308,486, filed on Feb. 9, 2022.

(51) Int. Cl. A61B 18/14; A61B 17/00; A61B 18/00; A61B 18/12

(52) U.S. Cl. CPC A61B 18/1492; A61B 17/00; A61B 18/1206; A61B 2017/00053; A61B 2018/0022; A61B 2018/00351; A61B 2018/00577

(57) ABSTRACT

A method may include providing an ablation system with (i) an ablation catheter having a plurality of antenna/sensor electrodes at a distal end; (ii) a plurality of ablation generators; (iii) a plurality of vector network analyzers (VNAs); (iv) a filtering system; and (v) a transmission system that individually couples each of the plurality of antenna/sensor electrodes to one discrete ablation generator and one discrete VNA. Each VNA may calculate high-frequency electrical parameters (HFEPs) for each antenna/sensor electrode. The method may further include navigating the ablation catheter to a target treatment region within a patient; capturing baseline HFEPs for each antenna/sensor electrode; positioning the ablation catheter; capturing updated HFEPs for each antenna/sensor electrode; identifying a first subset of antenna/sensor electrodes that are in contact with the target tissue and a second subset that is not; and selectively providing ablation signals to the first subset but not to the second subset.

### CROSS-REFERENCE TO RELATED APPLICATIONS

This application claims the benefit of U.S. Provisional Application Ser. No. 63/225,937, titled “Tissue Ablation and Lesion Assessment System,” filed on Jul. 26, 2021, and also claims the benefit of U.S. Provisional Application Ser. No. 63/308,486, titled “Tissue Ablation and Lesion Assessment System,” filed on Feb. 9, 2022.

This application incorporates the entire contents of the foregoing applications herein by reference.

### STATEMENT REGARDING FEDERALLY SPONSORED RESEARCH

This disclosure benefited from U.S. Governmental support under grant 1R43HL147787-01, awarded by the National Institutes of Health; accordingly, the Government has certain rights therein.

### TECHNICAL FIELD

Various implementations relate generally to medical devices and uses thereof for tissue ablation and lesion assessment.

### BACKGROUND

Ablation therapies may be used treat conditions such as cancer (e.g., by destroying cancer cells), cardiac arrhythmias (e.g., by ablating myocardial tissue to block errant electrical signals), pain (e.g., by destroying nerve cells or otherwise disrupting transmission of signals), and other conditions.

During an ablation procedure, one or more ablation delivery devices (e.g. a catheter or needle with one or more ablation electrodes or components) may be placed in target tissue (e.g., diseased tissue). Various imaging modalities may be employed to guide the placement, such as, for example, X-ray, magnetic resonance imaging (MRI), computed tomography (CT), ultrasound imaging, endoscopy, optical imaging, electromagnetic navigation, and mapping, etc.). Ablation energy may then be delivered to destroy target tissue. The ablation energy may include high-voltage DC or AC pulses in different waveforms (which may cause irreversible electroporation), pulsed electric fields (e.g., pulse field ablation (PFA)), radiofrequency (RF), thermal (heat or cooling/freezing from hot saline or water or from cryogenically cooled materials), microwave energy, ultrasound energy, laser energy. Substances may also be employed, in some implementations, to destroy target tissue—including, for example, chemicals, biologics, toxic agents, etc.

One specific condition that may be treated with ablation is atrial fibrillation—a common abnormal heart rhythm disorder that affects a large population of patients. In many cases, atrial fibrillation is understood to result from many different impulses within a patient's heart rapidly firing at once and in an uncoordinated manner, causing a chaotic rhythm in the atria, thereby preventing the patient's atria from efficiently pumping blood. Atrial fibrillation can result in poor circulation throughout the body and pooling and clotting of blood in the heart, which may increase stroke risk in patients suffering from this condition. For many patients, the rapid, uncoordinated firing of impulses is focused around the pulmonary veins in the left atrium; and one available treatment is to ablate tissue around the pulmonary veins to prevent impulses from propagating throughout the atria (one such procedure is pulmonary vein isolation or pulmonary vein antrum isolation (PVAI)).

Pulmonary vein ablation is not always straightforward or successful in addressing atrial fibrillation. One study that investigated specific failure modes identified (i) inability to position an ablation catheter and (ii) instability of the ablation catheter or inadequate tissue contact at the target side, or both, as contributing to 25% and 23%, respectively, of the reasons for either a lengthy or a failed ablation attempt. (Morady F, Adam Strickberger S, Ching Man K, et al. Reasons for prolonged or failed attempts at radiofrequency catheter ablation of accessory pathways. J Am Coll Cardiol. 1996 Mar. 27 (3) 683-689.)

Other studies have investigated the effects of incomplete ablation lines. One such study, found that even small gaps in an ablation line can allow pacing or atrial fibrillation signals to pass. Thus, incomplete tissue ablation may account for some ablation failures. (Melby S J, Lee A M, Zierer A, Kaiser S P, Livhits M J, Boineau J P, Schuessler R B, Damiano R J Jr. Atrial fibrillation propagates through gaps in ablation lines: implications for ablative treatment of atrial fibrillation. Heart Rhythm. 2008 September; 5 (9): 1296-301. doi: 10.1016/j.hrthm.2008.06.009. Epub 2008 Jun. 10. PMID: 18774106; PMCID: PMC2923579.)

In treating atrial fibrillation specifically, and in performing ablation to treat other conditions more generally, it may be advantageous to confirm contact between target tissue and a device that is to deliver ablation energy to the target tissue; moreover, it may be advantageous to assess in real time, the effect of the delivery of ablation energy on target tissue—for example, to confirm formation of a lesion having desired characteristics.

### SUMMARY

Described herein are devices, systems and methods for confirming contact between a device delivering ablation energy and target tissue, for ablating that target tissue, and for assessing and monitoring lesion formation that results from the ablation.

In some implementations, a method includes providing an ablation system. The ablation system may have (i) an ablation catheter having a plurality of antenna/sensor electrodes at a distal end; (ii) a plurality of ablation generators that are each configured to generate ablation signals; (iii) a plurality of vector network analyzers (VNAs); (iv) a filtering system having a plurality of channels; and (v) a transmission system. The transmission system may individually couple each of the plurality of antenna/sensor electrodes to one discrete ablation generator in the plurality of ablation generators and one discrete VNA in the plurality of VNAs, through one discrete channel in the plurality of channels in the filtering system. Each VNA in the plurality of VNAs may be configured to (A) transmit sensing signals across a spectrum of frequencies; (B) measure transmit power for the transmitted signals and measure received power for reflected-back signals; and (C) from the measured transmit power and measured received power, calculate high-frequency electrical parameters (HFEPs). Each channel in the filtering system may prevent ablation signals from interfering with sensing signals.

The method may further include navigating the ablation catheter to a target treatment region within a patient; with the plurality of VNAs, capturing baseline HFEPs for each antenna/sensor electrode; positioning the ablation catheter to be in at least partial contact with target tissue; with the plurality of VNAs, capturing updated HFEPs for each antenna/sensor electrode; identifying, from the updated HFEPs, a first subset of antenna/sensor electrodes that are in contact with the target tissue and a second subset of antenna/sensor electrodes that are not in contact with the target tissue; and with a subset of the ablation generators, selectively providing ablation signals to the first subset of antenna/sensor electrodes but not to the second subset of antenna/sensor electrodes.

In some implementations, the HFEPs include a first phase-reversal frequency parameter, F_{R1}, and a second phase-reversal frequency parameter, F_{R2}, for each antenna/sensor electrode. Identifying the first subset of antenna/sensor electrodes that are in contact with the target tissue may include determining that F_{R1} and F_{R2} parameters in the updated HFEPs are each greater than corresponding F_{R1} and F_{R2} parameters in the baseline HFPEs by a threshold frequency. In some implementations, the threshold frequency is 30 MHz; in some implementations, the threshold frequency is between 25 MHz and 50 MHz.

The method may further include, with the plurality of VNAs, capturing ablation-progress HFEPs; determining from the ablation-progress HFEPs whether ablation parameters (A) meet clinical objectives, (B) indicate a likelihood of adverse events, or (C) do not yet meet clinical objectives; and if ablation parameters are determined to meet clinical objectives, stopping the selective application of ablation energy; if the ablation parameters are determined to indicate a likelihood of adverse events either adjusting the selective application of ablation energy or stopping the selective application of ablation energy; and if the ablation parameters are determined to not yet meet clinical objectives, continuing the selective application of ablation energy.

In some implementations, determining that ablation parameters meet clinical objectives includes determining that F_{R1} parameters in the ablation-progress HFEPs are greater than corresponding F_{R1} parameters in the updated HFPEs by a first threshold frequency, and F_{R2} parameters in the ablation-progress HFEPs are greater than corresponding F_{R2} parameters in the updated HFPEs by a second threshold frequency. The first threshold frequency may be about 30 MHz, and the second threshold frequency may be about 20 MHz. In some implementations, determining that ablation parameters meet clinical objectives includes determining that F_{R1} parameters in the ablation-progress HFEPs are greater than corresponding F_{R1} parameters in the baseline HFPEs by a first threshold frequency, and F_{R2} parameters in the ablation-progress HFEPs are greater than corresponding F_{R2} parameters in the updated HFPEs by a second threshold frequency. The first threshold frequency may be about 30 MHZ, and the second threshold frequency may be about 50 MHz.

The ablation system may further include (vi) a cardiac mapping and navigation system; (vii) a controller; and (viii) and a switch that selectively couples or decouples the cardiac mapping and navigation and the transmission system. The controller may cause the switch to decouple the cardiac mapping and navigation system and the transmission system when the subset of ablation generators selectively provides ablation signals to the first subset of antenna/sensor electrodes. In some implementations, positioning the ablation catheter to be in at least partial contact with target tissue includes positioning the ablation catheter based on information received from the cardiac mapping and navigation system; in some implementations, positioning the ablation catheter to be in at least partial contact with target tissue includes positioning the ablation catheter based on information received from imaging equipment that is external to the ablation system.

The transmission system may include a plurality of coaxial cables, wherein a discrete coaxial cable couples each antenna/sensor electrode to a discrete channel in the filtering system. At least one antenna/sensor electrode in the plurality of antenna/sensor electrodes may be configured as a spiral antenna/sensor electrode having at least two turns. In some implementations, the ablation signals are radio-frequency ablation (RFA) signals; in some implementations, the ablation signals are pulse-field ablation (PFA) signals. PFA signals may include trains of high-voltage pulses of at least 1 KV, delivered at a field strength of at least 100 V/cm.

A method may include providing an ablation system having (i) an ablation catheter having a plurality of ablation electrodes and a plurality of antenna/sensor electrodes at a distal end; (ii) a plurality of ablation generators that are each configured to generate ablation signals; (iii) a plurality of vector network analyzers (VNAs); (iv) a filtering system having a plurality of channels; and (v) a transmission system. The transmission system may individually couple each of the plurality of ablation electrodes to one discrete ablation generator in the plurality of ablation generators and each of the plurality of antenna/sensor electrodes to one discrete VNA in the plurality of VNAs, through one discrete channel in the plurality of channels in the filtering system. Each VNA in the plurality of VNAs may be configured to (A) transmit sensing signals across a spectrum of frequencies; (B) measure transmit power for the transmitted signals and measure received power for reflected-back signals; and (C) from the measured transmit power and measured received power, calculate high-frequency electrical parameters (HFEPs). Each channel in the filtering system may prevent ablation signals from interfering with sensing signals.

The method may further include navigating the ablation catheter to a target treatment region within a patient; with the plurality of VNAs, capturing baseline HFEPs for each antenna/sensor electrode; positioning the ablation catheter to be in at least partial contact with target tissue; with the plurality of VNAs, capturing updated HFEPs for each antenna/sensor electrode; with the data processor, identifying, from the updated HFEPs, a first subset of ablation electrodes that are determined to be in contact with the target tissue and a second subset of ablation electrodes that are determined to not be in contact with the target tissue; and with a subset of the ablation generators, selectively providing ablation signals to the first subset of ablation electrodes but not to the second subset of ablation electrodes. In some implementations, each antenna/sensor electrode in the plurality of antenna/sensor electrodes is disposed between two ablation electrodes in the plurality of ablation electrodes.

### BRIEF DESCRIPTION OF THE DRAWINGS

FIG. 1A illustrates one exemplary implementation of a steerable catheter.

FIG. 1B illustrates an exemplary distal working tip of the steerable catheter of FIG. 1A, having an ablation electrode and a ring electrode.

FIG. 1C illustrates another exemplary distal working tip of the steerable catheter of FIG. 1A, having a circular ring on which may be disposed a plurality of electrodes.

FIG. 1D illustrates various waveforms through which pulsed field ablation (PFA) may be delivered.

FIG. 2A illustrates an exemplary dual-electrode antenna/sensor design.

FIGS. 2B-2C illustrate exemplary multi-electrode antenna/sensor designs.

FIG. 2D illustrates an exemplary spiral antenna/sensor electrode design.

FIG. 2E illustrates an exemplary base for the design of FIG. 2D.

FIG. 2F illustrates an exemplary spiral antenna/sensor electrode design.

FIG. 2G illustrates a distal section of an ablation catheter with multiple spiral antenna/sensor ablation electrodes and with ring electrodes.

FIG. 3 illustrates another exemplary steerable catheter system.

FIG. 4 illustrates a block diagram of an exemplary system for performing ablation.

FIG. 5 illustrates superposition of exemplary PFA ablation pulses with high-frequency sensing waves.

FIGS. 6A-6C illustrate high-frequency electrical properties (HFEPs) of an exemplary spiral antenna/sensor electrode.

FIG. 6D illustrates an exemplary time-domain representation of certain HFEPs.

FIGS. 7A-7C illustrate HFEPs of an exemplary focal ablation catheter.

FIGS. 8A-8D illustrate HFEPs of an exemplary dual-electrode catheter.

FIGS. 9A-9B illustrate exemplary correlations between HFEP shifts and contact force and area of electrode contact, respectively.

FIG. 10A illustrates an exemplary force sensor.

FIG. 10B illustrates the exemplary force sensor of FIG. 10A, disposed in a catheter.

FIGS. 11A-11D depict an ablation procedure that may proceed on tissue at multiple points simultaneously.

FIG. 12 depicts an exemplary method for ablating target tissue.

FIGS. 13A-13D illustrate exemplary variations of a multi-electrode circumferential catheter.

FIG. 14 illustrates an exemplary spiral antenna/sensor multi-electrode balloon catheter.

FIG. 15A illustrates an exemplary dual-electrode antenna/sensor (dipole) antenna.

FIGS. 15B-C illustrate exemplary applications of the dual-electrode antenna/sensor (dipole) antenna of FIG. 15A.

FIG. 16 illustrates an exemplary midfield antenna used in conjunction with spiral antenna/sensor electrode catheter.

FIGS. 17A-17B illustrate exemplary antennae-needle designs for tumor ablation.

### DETAILED DESCRIPTION

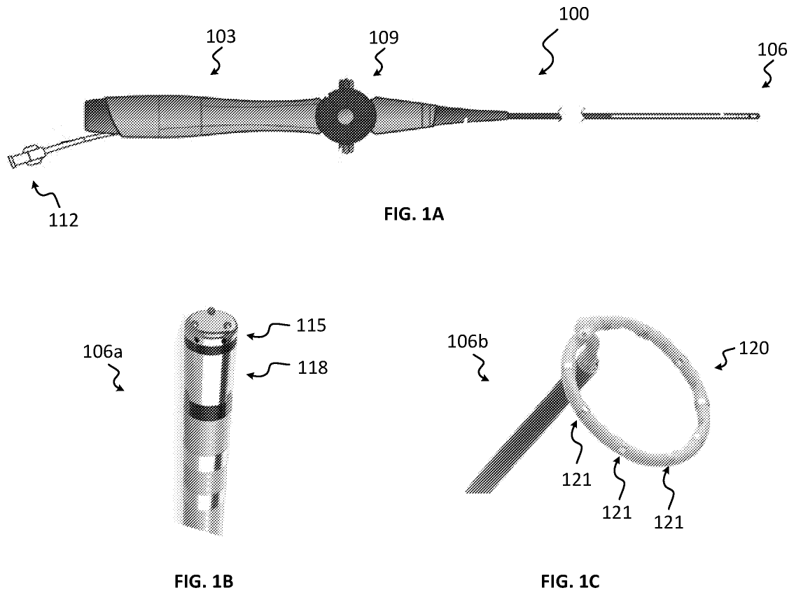

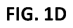

FIG. 1A illustrates one implementation of a steerable catheter 100, having a proximal handle 103; a distal working tip 106 that, during a procedure is disposed in a patient, at a target treatment region; steering controls 109; and one or more working channels or internal channels 112 (e.g., for supplying irrigation fluid or therapeutic compounds, for aspiration of fluids, for routing thermocouples, sensor signal conductors or ablation energy conducts, etc., to the distal working tip 106).

The distal working tip 106 may take various forms. For example, as shown in FIG. 1B, the distal working tip 106a may include an ablation electrode 115 and ring electrode 118 (which, in some implementations, may function as a ground). In some implementations, the electrode 115 and ring electrode 118 may be used for point-by-point or focal ablation procedures. As another example, as shown in FIG. 1C, the distal working tip 106b may include a circular ring 120 on which is disposed a plurality of electrodes 121. Such a distal working tip 106b may be employed in circular ablation applications, such as in procedures to isolate pulmonary veins.

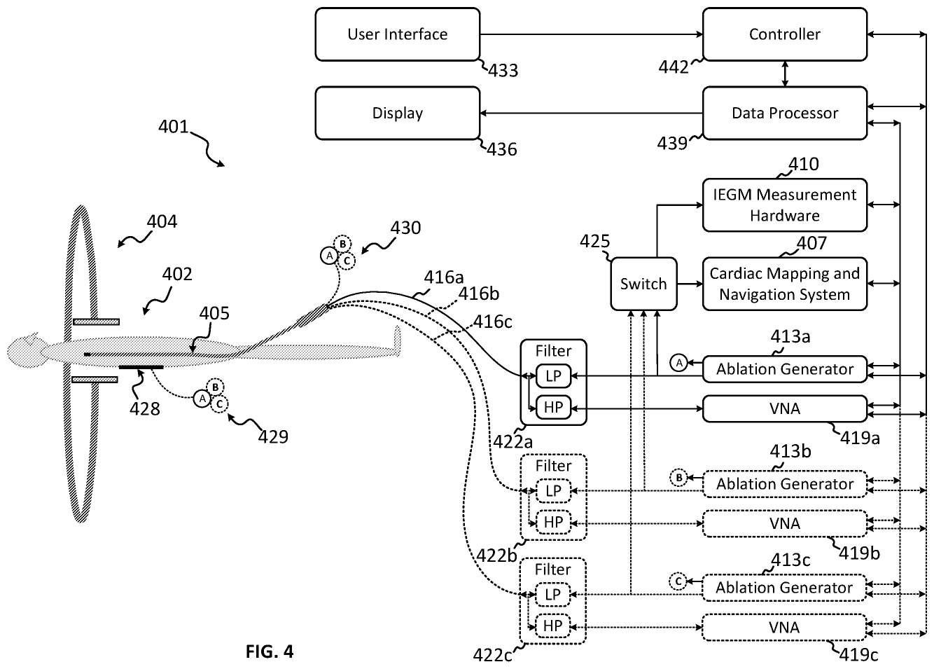

Various forms of energy may be delivered to a distal working tip 106 to selectively ablate target tissue. For example, with radiofrequency ablation (RFA), high-frequency sinusoidal alternating current signals may be delivered to target tissue of a patient via an ablation electrode (e.g., electrode 115 in FIG. 1B) and an external ground pad on the patient's skin (e.g., contact electrode 428 in FIG. 4). In some implementations, the signal may be between 300 and 900 KHz, delivered at 120-240 volts and 10-100 watts of power. In some implementations, the frequency is about 500 KHz, plus or minus about 25 KHz, (As used herein, “about” or “approximately” or “substantially” may mean within 1%, or 5%, or 10%, or 20%, 50% or 100% of a nominal value.)

As another example of ablation energy, thermal energy may be delivered (either heat, for example, with heated fluid, laser energy, microwave energy or electrical currents; or cold, with, for example, cryotherapy fluid).

As another example of ablation energy, high-voltage electrical pulses may be employed in pulsed field ablation (PFA) to induce cell death via irreversible electroporation (IRE). PFA may include trains of very short high-voltage pulses (e.g., about 100V to 50 KV, often 1-4 KV, delivered at a field strength of greater than 100 V/cm).

FIG. 1D illustrates various waveforms through which PFA may be delivered-including monophasic pulses 141a and 141b (e.g., pulses that are greater than about 10 μs), separated by a delay 142 (e.g., greater than about 100 μs, and, in some implementations, greater than about 250 ms); alternating monophasic pulses that include a first monophasic pulse 145a (e.g., a pulse that is greater than about 10 μs), a delay 146 (e.g., greater than about 100 μs, and, in some implementations, greater than about 250 ms), and another monophasic pulse 145b of opposite polarity; biphasic pulses that include a positive pulse 150a and negative pulse 150b (e.g., pulses that are greater than about 0.5 μs) within a given cycle 153, with phase delays 151 (e.g., greater than about 10 ns) and cycle delays 152 (e.g., greater than about 10 ns); or multiple packets 155a and 155b of biphasic pulses, each separated by a packet delay 156 (e.g., greater than about 250 ms).

Pulses such as those illustrated in FIG. 1D may be delivered to target tissue in various ways. For example, pulses may be delivered (e.g., to a patient's myocardium) by single-electrode or multi-electrode PFA catheters in multiple configurations (e.g., between two or more electrodes on the same catheter (e.g., an ablation electrode 115 and a grounding ring 118 in FIG. 1B, and between two or three electrodes 121 on circular catheter in FIG. 1C), between catheter electrodes (115 and 121) and grounding electrodes (428 in FIG. 4), between different electrodes on different catheters, between an electrode and an external grounding pad (e.g., on a patient's skin 428), etc.). And the pulses themselves could be any of the pulses illustrated in FIG. 1D (e.g., monophasic, biphasic, monopolar, bipolar, in packets, in some combination of the foregoing, with pulse parameters as small as nanoseconds or as high as a few hundred milliseconds, etc.).

### Desirability of Assessing Lesion Formation

Regardless of the type of ablation energy delivered, monitoring and assessing ablation progress is advantageous, if not necessary. Lesion formation may be monitored to determine lesion volume, depth of tissue ablated, extent of tissue ablation, lesion durability; and to characterize tissue type, predict potential adverse events, titrate ablation energy parameters to mitigate adverse events. It may be advantageous to regulate and/or maximize lesion formation and confirm irreversible lesion formation in real-time (e.g., while delivering ablation energies with one or more ablation modalities).

### Physiological Changes to Tissue During Ablation; can Measure/Assess by Looking at HFEPs

Different bodily fluids and tissues (e.g., blood, myocardium, scar tissue, fat, etc.) have different inherent dielectric properties. Moreover, during ablation, there may be a significant change to these dielectric properties-resulting, for example, from mobility of intra and extracellular water, dehydration, movement of ions or change in ion mobility, protein denaturation, cell membrane capacitance, changes in electrical charges, etc. Thus, by sensing these various dielectric properties, it can be possible to identify specific bodily fluids and tissue; and by measuring changes to various properties, progress of ablation can also be monitored.

In some implementations, dielectric properties of tissue can be sensed, and ablation progress tracked, by determining high-frequency electrical properties (HFEPs) of tissue. Specifically, target tissue can be analyzed for its response to high-frequency signals—for example, with a sensor that is designed as an antenna. HFEPs of a radio frequency (RF) or microwave antenna (e.g., an antenna designed to transmit and receive signals in the megahertz (MHz) to gigahertz (GHz) range). With such antennae, the HFEPs may be a function of the antenna design itself and dielectric and other properties of the medium (e.g., target tissue)—including, for example, conductivity, permittivity, and temperature of the medium (which may affect mobility of ions and molecules in the tissue, which, in turn, can affect dielectric properties).

When an antenna is placed in contact with tissue, its HFEPs change relative to when the antenna is not in contact with the tissue; moreover, when contact is maintained, the antenna response changes as dielectric properties of the contacting tissue changes. Given these points, a sensor designed as an antenna that is in contact with tissue being ablated can be used to detect and monitor changes to the tissue. In some implementations, this detecting and monitoring can be performed intraoperatively, allowing a clinical care provider who is performing an ablation procedure to monitor and quantify ablation procedure parameters (e.g., identify and confirm tissue type, confirm lesion formation, assess lesion extent or depth, etc.).

Specific HFEPs that may be relevant to monitoring tissue being ablated include reflection and transmission electrical properties of the antenna—including, for example, impedance, reflection coefficient, phase angle, voltage standing wave ration (VSWR), standing wave ratio (SWR), transmission attenuation, resonant frequency, phase-reversal frequency, Q factor, return loss, etc.—all of which may be a function of specific antenna design and properties of the medium (e.g., bodily fluid or tissue) surrounding the antenna. Some of the foregoing HFEPs may be measured using a vector network analyzer (VNA)—a device that can generate signals over a wide range of frequencies, measure the power of those signals transmitted to and received back (e.g., reflected back) from an antenna disposed in or around a particular medium, and use the measured transmitted and received signals to calculate various parameters. Other HFEPs may be measured using a time domain reflectometer, a time domain spectrometer, or other similar instruments in either or both of the frequency domain and time domain. In general, the HFEPs of a particular antenna—and by extension and calculated inference in some implementations, the dielectric properties of a medium in contact with the particular antenna—can be assessed or measured at one or more discrete frequencies, within a band or bands of frequencies, or over a wide sweep of the spectrum (e.g., in the MHz to GHz range).

In some implementations, an antenna-designed sensor may be implemented as both a sensor and an electrode configured to deliver ablation energy. With such a dual design, and with different frequencies for sensing HFEPs and delivering ablation energy (e.g., ablation energy delivered in high kilohertz (KHz) to low MHz, with sensing frequencies in a range of high MHz to low GHz), ablation frequencies may be filtered from the sensing frequencies, such that sensing and monitoring of lesion formation may occur simultaneously with ablation energy being delivered-on the same set of electrodes, intraoperatively.

### Exemplary Antenna Designs

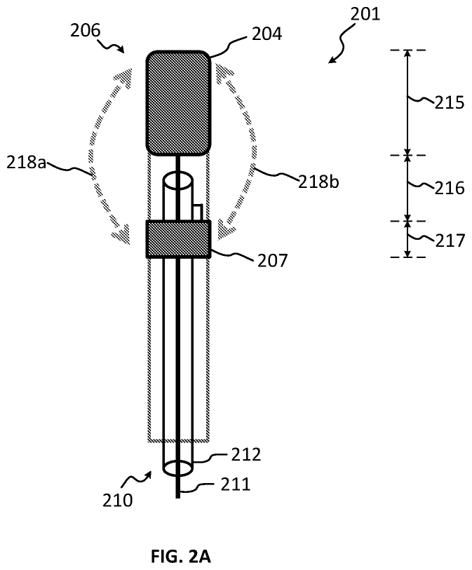

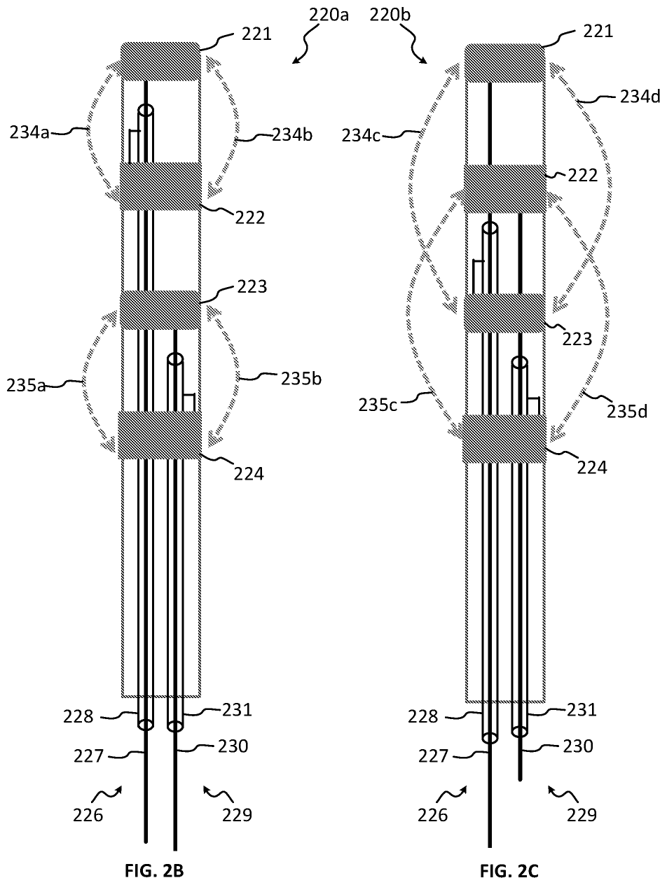

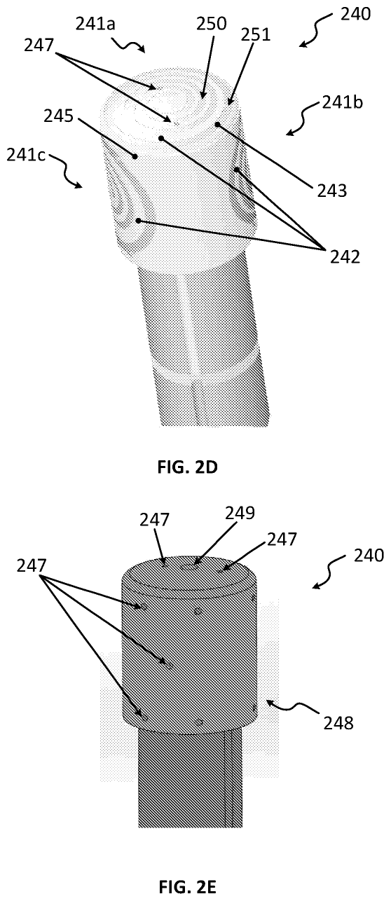

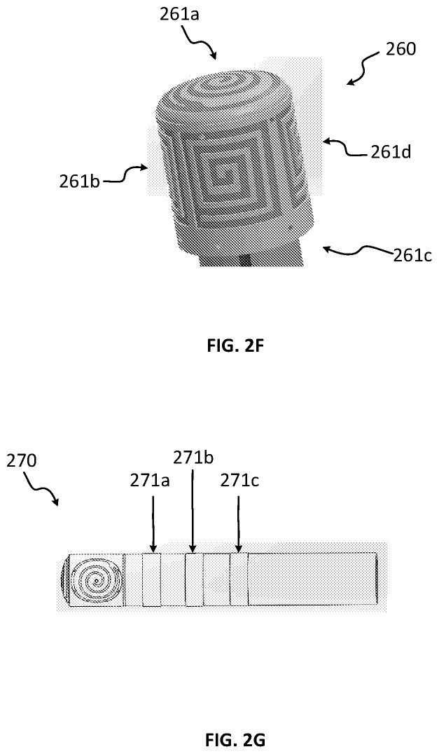

Various antenna designs that may be employed as ablation electrodes and sensors are now described. FIG. 2A illustrates an exemplary dual-electrode antenna design 201 that can be employed to simultaneously deliver ablation energy and sense and monitor lesion formation by monitoring HFEPs. As shown, the design 201 includes an ablation electrode 204 at a distal tip 206 and a ring electrode 207; the electrode 204 may be coupled to an inner conductor 211 of a coaxial cable 210, and the ring electrode 207 may be coupled to a shield 212 of the coaxial cable. Ablation energy (e.g., in the form of RFA or PFA) may be delivered to the ablation electrode 204 (with the ring electrode 207 or grounding pad 428 providing a return path), while HFEPs may be monitored across the same electrodes 204 and 207 at a higher-frequency signal than the ablation energy is delivered.

The dual-electrode antenna design 201 may be compatible with other ablation modalities; for example, the design 201 could be employed with a cryoablation system, where the electrodes 204 and 207 could be used just for monitoring.

In some implementations, a gap (e.g., length 216) of about 3 cm or less may separate the ablation electrode 204 from the ring electrode 207. In some implementations, the ablation electrode 204 is about 1 mm in length (as measured along a longitudinal axis of the catheter—e.g., length 215); in other implementations, the ablation electrode 204 may be about 3 mm in length. The ring electrode may be less than about 2 mm in length (e.g., length 217). A sensing zone may correspond to the high-frequency field lines 218a and 218b shown in FIG. 2A (as shown in various figures, high-frequency sensing field lines, such as field lines 218a and 218b, are illustrated on either side of electrodes, but the reader will appreciate that, depending on the medium in which the antenna design is disposed, these field lines may extend in three dimensions, around the circumference, or a portion thereof, from the corresponding electrodes).

FIG. 2B illustrates another exemplary multi-electrode antenna design 220a. As shown, the design 220a includes four electrodes—a first electrode 221, a second electrode 222, a third electrode 223 and a fourth electrode 224. A first coaxial conductor 226 is coupled to the first and second electrodes 221 and 222—specifically, as shown, an inner conductor 227 is coupled to the first electrode 221 and the shield 228 is coupled to the second electrode 222; and a second coaxial conductor 229 is coupled to the third and fourth electrodes—specifically, as shown, an inner conductor 230 is coupled to the third electrode 223 and the shield is coupled to the fourth electrode. A first set of sensing field lines 234a and 234b, as shown, may extend between the first electrode 221 and second electrode 222; and a second set of sensing field lines 235a and 235b, as shown, may extend between the third electrode 223 and fourth electrode 224.

In some implementations, the antenna design 220a may be configured as a dipole antenna (e.g., based on the signals incident on the coaxial conductors 226 and 229); in other implementations, the antenna design 220a may be configured as two independent monopole antennas.

FIG. 2C illustrates another multi-electrode antenna design 220b, which is a variation of the design 220a. Specifically, the coaxial cables are coupled differently—such that the first coaxial cable 226 is coupled to the first electrode 221 and third electrode 223, and the second coaxial able 229 is coupled to the second electrode 222 and the fourth electrode 224—resulting in sensing field lines 234c and 234d, and overlapping sensing field lines 235c and 235d.

In some implementations, antenna designs such as design 220a or 220b may facilitate sensing and monitoring a greater area of tissue than may be possible with only two electrodes-thereby giving a caregiver performing an ablation procedure more information about the tissue being ablated or the progress of the ablation. Moreover, designs such design 220a and 220b may provide greater flexibility with respect to delivery of ablation energy. For example, ablation energy may be delivered over multiple sets of electrodes simultaneously, which may, in some implementations, accelerate overall procedure time by enabling ablation of multiple target areas simultaneously or ablation of larger areas than would be otherwise possible.

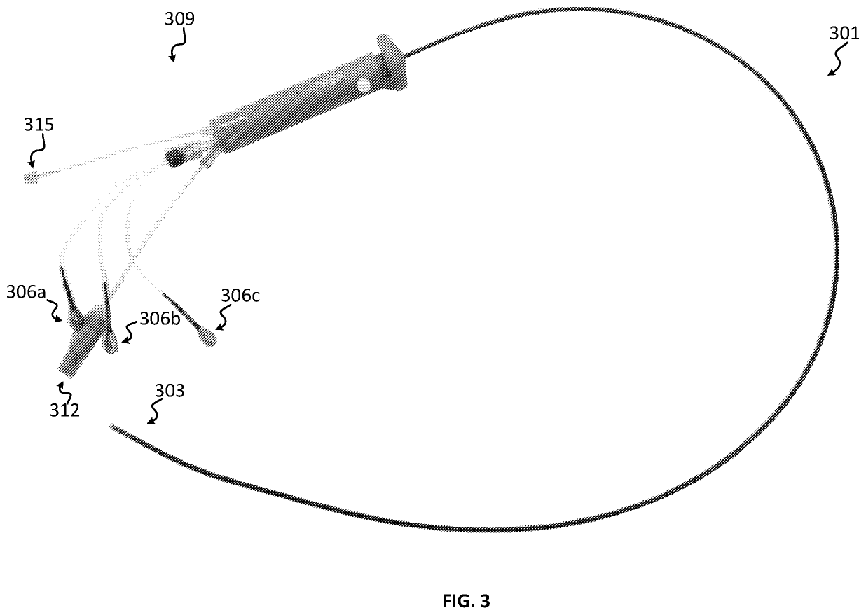

FIG. 2D illustrates another design 240 for a distal tip or ablation electrode of a catheter. As shown, the design 240 includes three spiral antennas 241a, 241b and 241c—with one antenna 241a disposed on a distal tip of the catheter, and two spiral antennas 241b and 241c disposed on the sides of the catheter (e.g., 180 degrees apart, as shown). Each antenna 241a, 241b and 241c may be coupled to a low-noise coaxial cable (not shown) configured as a high-frequency transmission line that, in some implementations, is capable of propagating signals in the DC to about 10 GHz range (e.g., between an ablation signal generator and a sensing equipment on one end, and target tissue to be ablated on the other end). The coaxial cables may run internally along the length of the catheter and may terminate at connectors 306a, 306b and 306c at a proximal end (see FIG. 3). With antennas 241a, 241b and 241c disposed as shown, at least one antenna (and often two) will be in contact with target tissue—as will be further illustrated and described.

Each spiral antenna 241a, 241b and 241c may comprise a spiral conductor 242 on a dielectric substrate 243. A common ground electrode 245 may be disposed around the spiral antennas 241a, 241b and 241c. In some implementations, ports 247 may be provided that are coupled to lumens that terminate outside of the catheter—for example, to facilitate irrigation of or aspiration from a target treatment region adjacent a distal tip of the catheter.

With reference to FIG. 2E, a base 248 of the electrode 240 may, in some implementations, be made out of a ceramic dielectric, such as alumina (Al₂O₃) or another dielectric with a thermal conductivity of greater than about 5 Watts/meter-Kelvin (W/mK). In some implementations, the dielectric constant may be less than 100; the dielectric loss tangent may be less than 0.001 at frequencies in the MHz to GHz; the dielectric breakdown voltage may be greater than 100 V/mm; the electrical resistivity may be greater than 10 ohms/cm. In some implementations, the base 248 may further include or be made from Aluminum Nitride (AlN), silicon carbide, industrial diamonds, plastic (e.g. polytetrafluoroethylene (PTFE)), etc.

In some implementations, the base 248 includes ports 247 coupled to lumens (not shown) that can be configured for saline flush irrigation or closed-loop irrigation. Inside the base 248, other lumens/channels (not shown) can be provided, for example to route coaxial cables to the spiral antennas 241a, 241b or 241c, to the ground electrode 245, or to other sensors (not shown) such as thermocouples and contact force sensing hardware (e.g., fiber optic force sensors, electromagnetic force transducers, etc.).

In some implementations, with reference back to FIG. 2D, the spiral antennas 241a, 241b and 241c and the ground electrode 245 may include one or more metals disposed (e.g., deposited or plated) on the base 248. Each spiral antenna 241a, 241b, and 241c may, in some implementations, include greater than ½ of a turn, and typically may include two to four turns, with a gap 250 of greater than about 0.001″ (25 microns) between turns, and an outer gap 251 (between the spiral antenna 241a and the ground electrode 245) also of greater than about 0.001″. Metal thickness for the spiral antennas 241a, 241b and 241c and the ground electrode 245 may, in some implementations, be greater than about 0.5 microns (e.g., configured to facilitate greater skin depth that about ⅕^{th} of the metal thickness at 500 KHz ablation frequencies). More typically, the metal thickness may be between 10-100 microns, and may include one or more conductive metals, such as gold (Au), silver (Ag), nickel (Ni), copper (Cu), molybdenum (Mo), manganese (Mn), titanium (Ti), niobium (Nb), chromium (Cr), Nickel (Ni), etc. The metal may be completely disposed, or disposed in layers, to optimize substrate bonding. The base 248 may include one or more dielectric materials that have been selected to facilitate or optimize metal bonding, dielectric properties, thermal properties, etc. The metal may be deposited using sputter coating, vapor deposition, electroplating, or any other suitable processes.

FIG. 2F illustrates another exemplary design 260 for a distal tip or antenna/sensor electrode that includes multiple spiral antennas 261a, 261b, 261c and 261d. As shown, the design 260 may include spiral antennas having different configurations-such as the antennas 261b, 261c and 261d, which have a squared-off spiral design. Moreover, as shown, more than just two antennas may be disposed on the circumferential side of the design 260.

FIG. 2G illustrates another exemplary design 270 for a distal end-which, as shown, includes the multi antenna/sensor electrode 240 of FIG. 2D, but with three additional electrodes 271a, 271b and 271c. In some implementation, the electrodes 271a, 271b and 271c are ring electrodes that may be 1.5 to 3 mm in length (when measured relative to a longitudinal axis of the catheter). In some implementations, the inclusion of such electrodes 271a, 271b and 271c can facilitate use of the design 270 with existing catheter navigation systems. Moreover, the electrodes 271a, 271b and 271c in conjunction with 240 may provide additional monitoring, pacing and sensing capabilities when coupled with other teachings of this disclosure.

FIG. 3 illustrates another implementation of a steerable catheter system 301 that can be employed to ablate target tissue while simultaneously facilitating monitoring of the ablation process. The catheter system 301 includes a steerable distal tip 303 that is configured for navigation into internal lumens or cavities of a patient. The distal tip 303 may include one or more electrodes configured as antennas, as just described. At the distal tip 303, the catheter system 301 may include other sensors and components (not shown), including, for example thermocouples, electromagnetic sensors (for magnetic navigation), and force transducers, which may be connected by appropriate transmission lines along the length of the catheter to connectors 306a, 306b and 306c at a proximal end 309 of the catheter system. Inclusion of various sensors may enable the catheter system 301 to integrate with readily available equipment commonly found in catheterization laboratories, while also facilitating simultaneous ablation and monitoring and assessment of lesion formation. Other connectors and ports may be provided, which are accessible from the proximal end 309. For example, intracardiac electrogram (IEGM) electronics and/or mapping electronics may be provided in the catheter system 301, and a connector 312 may be provided at the proximal end 309 with which to couple external analysis and control equipment to such systems. Moreover, other working channels and ports may be provided, such as port 315 (e.g., for saline flushing systems or aspiration systems).

### Exemplary Overall System

FIG. 4 illustrates a block diagram of an exemplary system 401 for performing an ablation procedure on a patient 402 and monitoring progress of that procedure (e.g., monitoring, in real time, lesion formation). Ancillary to the system 401 may be an external imaging system 404, such as x-ray, fluoroscopy or other imaging equipment readily available in a catheter laboratory for assisting a physician in positioning an ablation catheter 405 in the patient 402. In addition to the external imaging system 404, the system 401, as shown, includes a cardiac mapping and navigation system 407 and IEGM measurement hardware 410.

An antenna/sensor electrode (e.g., the spiral antenna 241a shown in FIG. 2D) on a distal tip of the catheter 405 may be coupled to an ablation generator 413a via a dedicated coaxial cable 416a. To facilitate simultaneous ablation (e.g., PFA, RFA or another ablation modality) and sensing and assessment of formation of a corresponding lesion, the dedicated coaxial cable 416a may also couple the antenna/sensor electrode to a VNA 419a (or VNA channel), through a filter 422a. As shown, the filter 422a may include a low-pass (“LP”) component and a high-pass (“HP”) component—wherein the HP component passes only high-frequency assessment signals (e.g., in the range of MHz to low GHz (e.g., 2 MHz to 2 GHz, or 2 MHz to 20 GHz, or 10 MHz to 5 GHZ, etc.) through to the VNA while blocking lower-frequency ablation signals from the VNA; and the LP component may pass lower-frequency cardiac mapping, navigation and IEGM signals through to the cardiac mapping and navigation system 407 and IEGM measurement system 410.

The filter 422a may enable simultaneous propagation of DC-GHz signals—for example, high frequency assessment signals; low frequency ablation signals (PFE/IRE/PFA and RFA energies, etc.), along with low frequency signals for IEGM measurement, navigation, impedance measurement, etc. In some implementations, the LP filter elements are included on either or both of the ground or return path and on the positive portion of a circuit at the low frequency end (ablation generator, etc.); and the HP filter elements are included on either of both of the ground or return path and the positive portion of a circuit (e.g., prior to the VNA). Such a configuration can facilitate separation of high frequency signals from the low frequency signals, enabling stable and consistent high-frequency monitoring during ablation.

In some implementations, a single filter circuit configuration may be employed for monitoring RFA and PFA procedures. The LP filter may have an isolation of greater than about 20 dB at frequencies greater than about 1 MHz and an insertion loss of less than about 1 dB at frequencies less than about 1 MHz. The HP filter may block frequencies less than about 1 MHz with greater than 20 dB isolation and may have an insertion loss of less than about 1 dB at frequencies greater than about 1 MHz, particularly, for example, in the measurement range of 10 MHz to 10 GHz.

Depending on the specific ablation modality, a switch 425 or attenuator may be provided to protect the cardiac mapping and navigation system 407 and IEGM measurement system 410 from active ablation signals (e.g., the switch may be opened when ablation signals are active that may be harmful to these systems).

Depending on the ablation modality, a common return path for ablation signals may be provided via an external contact 428 on the patient's skin (via connection 429); alternatively, a common return path for ablation signals may be provided via an electrode (e.g., a ring electrode, such as ring electrode 271a, 271b or 271c in FIG. 2G) on the catheter 405 (e.g., by coupling the common return path to a connection 430 with a corresponding conductor associated with the electrode.

In some implementations, as shown, the low-frequency input and output for each antenna/sensor electrode can be kept separate for various functions, such that low-frequency hardware (ablation, navigation, IEGM measurement, impedance measurement, etc.) can be individually connected to each antenna-sensor component and low frequency electrical properties and ablation functions monitored and controlled individually. In other implementations, the low frequency input and output of each antenna/sensor electrode may be coupled to a common low-frequency input/output (such that the high-frequency signals for the antennae/sensors electrodes are discrete at high frequencies, but the low frequency signals may be combined into a single input and output). In the latter implementations, it may be possible to reduce the number of conductors within the catheter 405 by only isolating those conductors that must be discrete. For example, with the right filtering in place, it may be possible to couple certain sensors (e.g., electromagnetic navigation-related sensors, thermocouples) and other components that are not associated with ablation to the ablation generator(s) 413a, 413b and 413c, IEGM measurement hardware 410 and cardiac mapping and navigation system 407—since, in many implementations, these functions are performed at frequencies less than about 1 MHZ (and would therefore not be interference between such signals and higher-frequency signals (high MHz to GHz signals) used for monitoring and assessing ablation. On the other hand, with ablation modalities at much higher frequencies (e.g., 100 MHz—which may be required for nanosecond pulse width PEF/PFA pulses), appropriate band-selective filters may be employed to separate ablation frequencies and the sensing frequencies. In addition to configuring the filter(s) 422a, 422b and 422c to provide appropriate filtering and signal separation, antenna/sensor electrodes themselves can be designed to have sensing frequencies that exclude ablation frequencies.

Overall control of the system 401 may be managed through a user interface 433 and display 436, with computing and control functions being provided by a data processor 439 and controller 442.

Optionally, in some implementations, multiple ablation generators and VNAs (or VNA channels) may be employed For example, a second ablation generator 413b may be coupled to a dedicated coaxial cable 416b that couples to a dedicated antenna/sensor electrode at a distal end of the catheter 405 (e.g., antenna/sensor electrode 241b in FIG. 2D). Again, the coupling may be through a filter 422b, such that high-frequency signals can be filtered and routed to a dedicated VNA 419b; and low-frequency signals may be (optionally) routed to the cardiac mapping an navigation system 407 and/or the IEGM measurement hardware 410. Other ablation generators and VNAs may be provided in a similar manner (e.g., ablation generator 413c and VNA 419c, via dedicated coaxial cable 416c, via a filter 422c—which may be coupled to another antenna/sensor electrode at the distal end of the catheter 405, such as the antenna/sensor electrode 241c in FIG. 2D).

In implementations such as those just described, it may be possible to separately monitor the HFEPs of each antenna/sensor electrode separately; thus, the system 401 may simultaneously provide information about multiple locations within an ablation region, while the ablation is being performed. Such implementations may facilitate a reduction of procedure time, greater control over lesion formation during the procedure, and confirmation that the lesion formation is sufficient to meet clinical objectives.

In some implementations with multiple ablation generators 413a, 413b and/or 413c, each ablation generator may ablate using the same modality (e.g., PFA, RFA, etc.); in other implementations, multiple modalities of ablation may be available within the same system 401. One or more ablation generators 413a, 413b or 413c may be a high-wattage PFA, PEF, IRE, or RFA signal generator, and the caregiver can select/switch between the ablation modality as required. The output of each generator 413a, 413b or 413c may be controlled by the controller 422, which may be a proportional, on-off, or other suitable controller configured to titrate ablation energies to accomplish clinical objectives of the lesion formation. The controller 422 may automatically reduce or stop the ablation energy if an unsafe condition is detected and when completion of lesion formation is detected. The controller 422 may also be configured to provide an alert when desired lesion depth or extent is attained or if any preventive or corrective action is required. In some implementations, the ablation generator(s) 413a, 413b and 413c may be directly controlled by the controller 422; in other implementations, an accessory regulator device or system (not shown) may be employed within or external to the system 401.

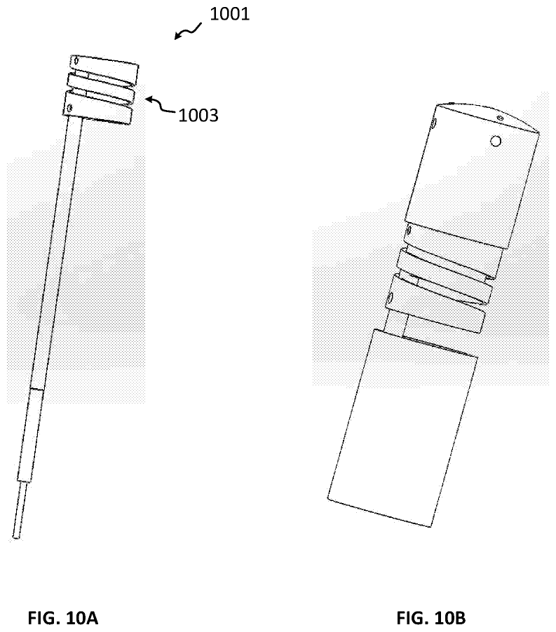

In some implementations, other sensors may be incorporated into the system 401. For example, a dedicated force sensor may be provided (see, for example, FIGS. 10A and 10B). As another example, various fiber optic cable-related sensors may be incorporated—specifically, for example, a Mach Zehnder e-field sensor (disposed, for example, in close proximity to one or more antenna/sensor electrodes or between two antenna/sensor electrodes; and corresponding optical transducer equipment may be routed to the data processor 439). Other sensors may be provided.

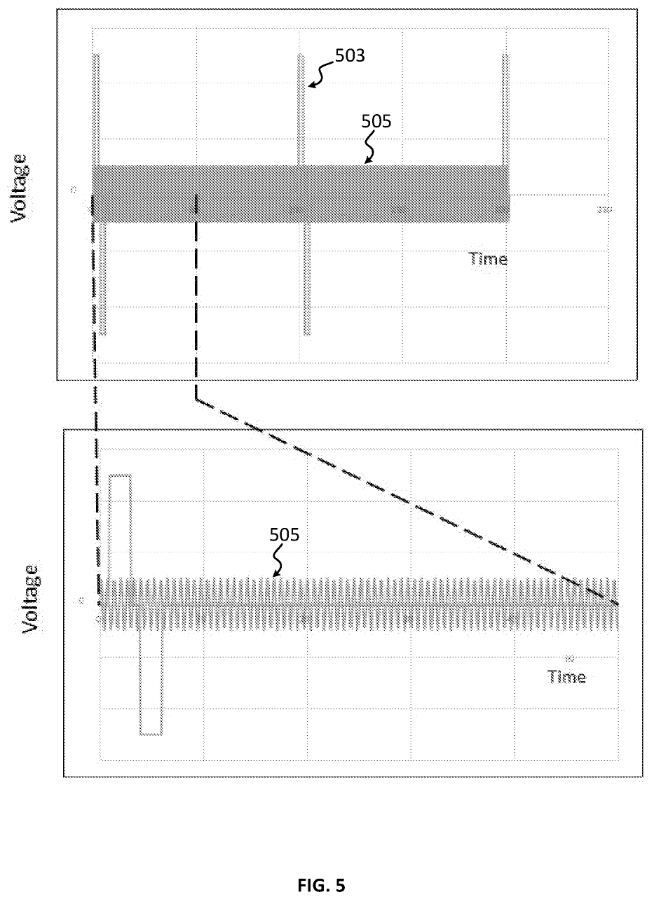

FIG. 5 illustrates an exemplary superposition of PFA ablation pulses 503 with a high-frequency sensing wave 505. As illustrated in FIG. 5, the frequencies of the two waveforms differ greatly, and could be easily filtered as described above.

### Exemplary Ablation Procedures

An exemplary ablation procedure is now described—in particular, a method by which progress of the procedure may be monitored. First, a system such as the system 401 of FIG. 4 may be configured. Prior to the catheter 405 (e.g., an ablation catheter such as the catheter 301 of FIG. 3) being disposed in a patient 402 undergoing the procedure, all relevant channels of the VNA (e.g., VNA 419a, and VNA 419b and/or VNA 419c) may be calibrated (e.g., checked and/or adjusted at open, short, and loaded conditions, in a manner that simulates operation within the patient 402). Appropriate calibration equipment may be employed during calibration (e.g., filter circuits, extension cables and coaxial cable electrical equivalents which are in the catheter, such that the VNA 419a is electrically calibrated to the distal end of the catheter 405 electrically). Custom calibration units may be designed and used for accurate and automated calibrations. In some implementations, various calibration parameters may be loaded into the VNA 419a and automatically loaded based on the specific catheter 405 connected in the system 401.

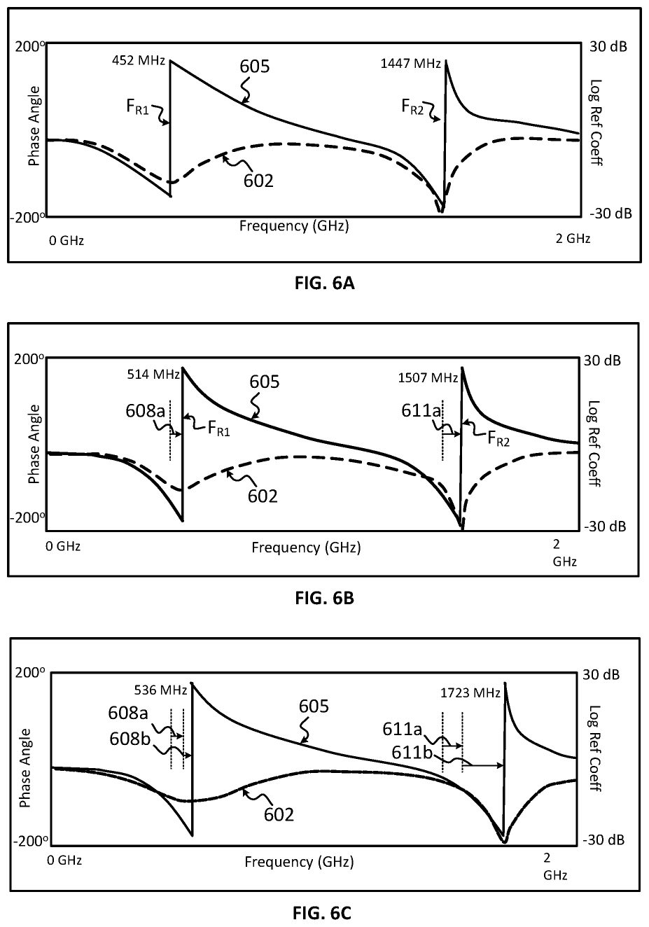

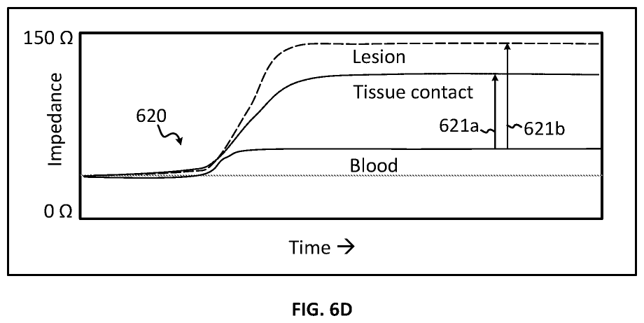

After calibration, the catheter 405 may be coupled to the filter 422a; and a physician may guide the catheter 405 into position in the patient 402, for example, with real-time assistance from imaging equipment 404 and from the cardiac mapping and navigation system 407 (e.g., with reference to the display 436 and using manual navigation and the user interface 433). Once the catheter 405 is in position, information from the VNA 419a may be processed by the data processor 439 and presented on the display 436, to assist the physician in preparing to perform an ablation. Reflection impedance properties may be measured and captured by the VNA 419a, processed by the data processor 439, and presented on the display 436. The log of a reflection coefficient and a phase angle may be particularly useful in guiding the physician in the ablation procedure, as is now described with reference to FIG. 6A-6C.

FIGS. 6A-6C illustrate exemplary HFEPs of on a spiral antenna/sensor electrode, and changes thereto, during a PFA ablation procedure. FIG. 6A illustrates exemplary HFEPs during a frequency sweep of about 10 MHz to 2 GHz of a specific spiral antenna/sensor electrode (e.g., like the antenna/sensor electrode 241a of FIG. 2D. The amplitude of the logarithm of the reflection coefficient 602 and the phase angle 605 are first measured with the antenna/sensor electrode disposed only in blood (i.e., not contacting tissue). As shown in this example, the phase angle 605 has two resonant frequencies (phase-reversal frequencies): F_{R1}, at 452 MHz and F_{R2} at 1447 MHz.

FIG. 6B illustrates HFEPs of the same spiral antenna/sensor electrode as in FIG. 6A, but with that antenna/sensor electrode contacting myocardium tissue. As shown, F_{R1} shifts to 514 MHZ—an increase 608 of 62 MHZ—and F_{R2} shifts to 1507 MHz—an increase 611 of 60 MHz. As illustrated in FIG. 6B, then, in some implementations, an increase in F_{R1} and F_{R2}, relative to the values of F_{R1} and F_{R2} in blood (i.e., without contact with tissue) can indicate contact with tissue. Also as shown in FIGS. 6A and 6B, dips in the logarithm of the reflection coefficient 602 may correspond to the phase-reversal frequencies F_{R1} and F_{R2}—and thus these dips may provide confirmation of the phase-reversal frequencies F_{R1} and F_{R2}—particularly, for example, in implementations that may be noisier than the exemplary implementation depicted in FIGS. 6A and 6B.

Different antenna/sensor electrode designs will have different HFEPs; and responses may also differ depending on tissue type. Thus, F_{R1} and F_{R2} may be different, based both on antenna/sensor electrode design and tissue and/or bodily fluid in contact with the antenna/sensor electrode. The changes, however, in going from blood-only contact to tissue contact may be representative. That is, for many antenna/sensor electrode designs, a change of about 25-100 MHz (and more particularly, in some implementations, a change of about 50-75 MHz) may be observed when an antenna/sensor electrode contacts tissue, relative to blood-only contact.

FIG. 6C illustrates HFEPs of the same spiral antenna/sensor electrode as in FIGS. 6A and 6B, but with ablation underway. Specifically depicted in FIG. 6C is an ablation procedure in which a bipolar-biphasic PFA voltage of 600V is applied between a spiral antenna/sensor electrode (e.g., spiral 242 of the antenna/sensor electrode 241a of FIG. 2D) and a ground plane of the electrode (e.g., ground electrode 245 shown in FIG. 2D). As depicted, during ablation, F_{R1} increases to 536 MHz and F_{R2} increases to 1723 MHz—an increase in F_{R1} of 608b, beyond the increase 608a, relative to blood-only contact; and an increase in F_{R2} of 611b, beyond the increase 611a, relative to blood-only contact. These second increases 608b and 611b may confirm lesion formation. In particular, in some implementations, permanent increases 608a plus 608b in F_{R1} of greater than about 50 MHz and permanent increases 611a plus 611b in F_{R2} of greater than about 90 MHz can indicate a durable and irreversible lesion—which, in some implementations, corresponds to clinical objectives of an ablation procedure.

The shifts in F_{R1} and F_{R2} may be explained as follows: F_{Rx}=½π\sqrt{LC}, during PFA, the applied voltage pulses cause cell wall disruption, reducing the cell membrane capacitance, hence increasing the FRY (e.g., as illustrated in FIGS. 6A-6C).

As illustrated in FIG. 6D, the HFEPs of an antenna/sensor electrode may also be measured in the time domain—for example, using a time-domain spectrometer (TDS) or a VNA. Such a measurement may be done in isolation, or it may be made in addition to the frequency-domain measurements illustrated in and described with reference to FIGS. 6A-6C. As shown in FIG. 6D, impedance 620 of the antenna/sensor electrode in blood, in contact with target tissue, and during and after ablation may change. In particular, as shown, the impedance 620 may change from 650-75Ω in blood, to 95-100Ω (an increase 621) when the antenna/sensor electrode is in contact with tissue. During PFA, the impedance 620 may gradually increase to 110-130Ω (an increase 622), which can confirm e-field deposition and lesion formation. Specific values depicted in FIG. 6D are only representative. Characteristic impedance values and changes during the procedure are a function of antenna/sensor design, tissue/bodily fluid type, and ablation modality used; and specific changes for given designs, tissues/fluids, and modalities may be benchmarked to facilitate assessment and monitoring of lesion formation and to predict and avoid adverse events.

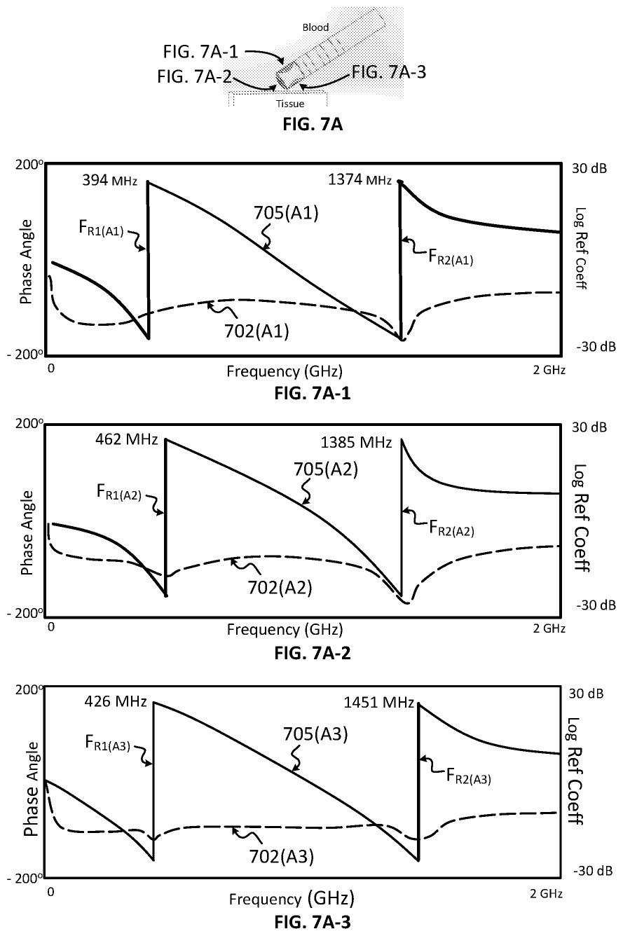

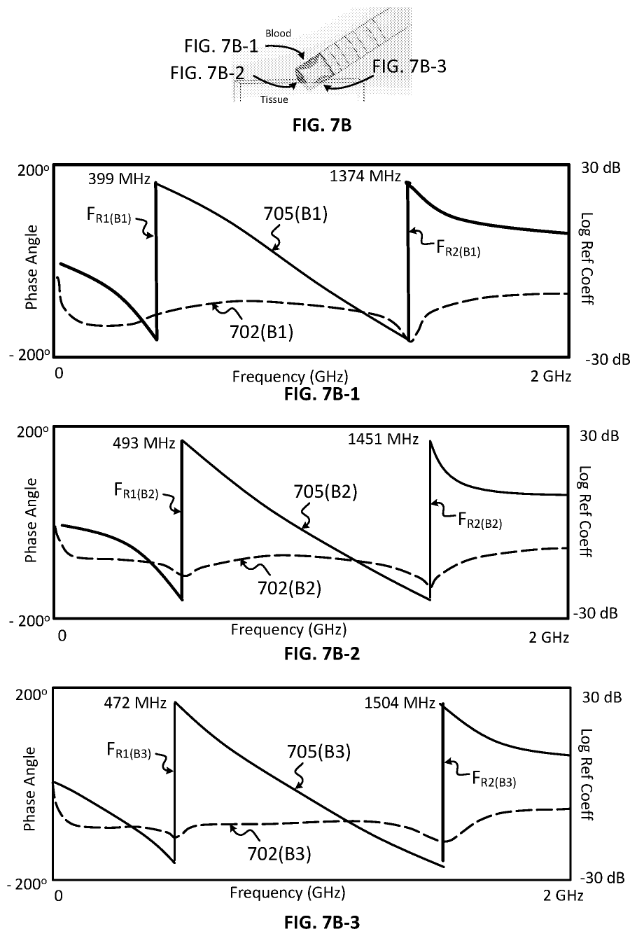

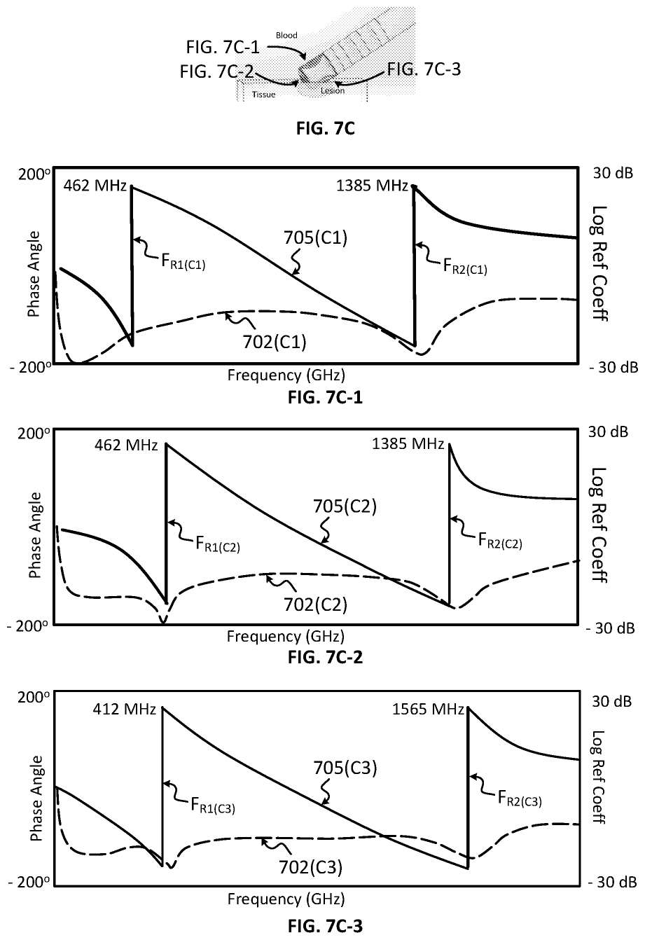

FIGS. 7A-7C illustrate exemplary HFEPs of a focal ablation catheter having multiple antenna/sensor electrodes (e.g., like the design 270 shown in FIG. 2G), and changes thereto, during a RFA ablation procedure. As in FIG. 6A-6C, phase angle 705 and the logarithm of the reflection coefficient (i.e., return loss) 702 are monitored by frequency sweep measurements in the range of about 1 MHz to 2 GHz; and resonant frequencies, F_{R1} and F_{R2} (i.e., phase-reversal frequencies) are noted. In the figures, each parameter is parenthetically referenced to the appropriate antenna/sensor electrode (e.g., side electrode away from tissue (1), distal electrode (2), and side electrode facing tissue (3)) and to the orientation of the electrode relative to the tissue (e.g., in blood (A), contacting tissue (B), and contacting and ablating tissue (C))—thus, the parenthetical “(A2)” is associated with the distal electrode in blood; the parenthetical “(C3)” is associated with the side electrode facing the tissue-when it is in contact with the tissue and ablating it; etc.

As illustrated in and described with reference to FIGS. 6A-6C, the HFEPs are different when corresponding antenna/sensor electrodes are contacting blood only, contacting tissue, or ablating the tissue. Thus, by monitoring these parameters for each antenna/sensor electrode, a physician controlling an ablation procedure can infer both a position of each antenna/sensor electrode and monitor progress of the ablation procedure. The HFEPs are merely representative; actual parameters will vary based on antenna/sensor design, type of tissue and/or bodily fluid, and effect of the specific modality of ablation (e.g., PFA, RFA, cyro, etc.) on the target tissue. Two resonant frequencies are shown in FIGS. 7A-7C; but in other implementations, additional resonant frequencies may be present at different (e.g., higher frequencies).

FIG. 7A depicts baseline measurements for HFEPs for the three antenna/sensor electrodes in blood—specifically, FIG. 7A depicts the distal tip of the catheter in blood above the tissue; FIG. 7A-1 depicts HFEPs for the outer side antenna/sensor electrode; FIG. 7A-2 depicts HFEPs for the distal antenna/sensor electrode; and FIG. 7A-3 depicts HFEPs for the inner side (i.e., facing tissue) antenna/sensor electrode.

The measurements depicted in FIGS. 7A, 7A-1, 7A-2 and 7A-3 may provide a baseline from which a physician and/or an ablation system can determine: (i) orientation of electrode-tissue contact, (ii) area of electrode in tissue contact, (iii) contact force, (iv) type of tissue in contact; and during ablation, (v) extent of lesion formed, and (vi) the potential for adverse events occurring (e.g. microbubble formation, etc.)—which can be used to tailor the ablation parameters for creating and confirming a durable lesion, safely and reducing collateral injuries.

As depicted in FIGS. 7B, 7B-1, 7B-2 and 7B-3, upon contacting tissue (e.g., myocardium), F_{R1(B2)} and F_{R2(B2)}, and F_{R1(B3)} and F_{R2(B3)} increase (e.g., proportionally to the extent of contact). As shown in this design and orientation, F_{R1(B2)} increases relative to F_{R1(A2)} and F_{R1(B3)} increases relative to F_{R1(A3)} by about 30-50 MHz (in other similar implementations, this increase may typically be about 40-60 MHZ), and F_{R2(B2)} increases relative to F_{R2(A2)} and F_{R2(B3)} increases relative to F_{R2(A3)} by about 50-70 MHz (in other similar implementations, this increase may typically be about 50-120 MHz).

As shown, since the distal tip is contacting the tissue at an oblique angle, and the top side sensor is not in contact with the tissue; hence, there is little change in F_{R1(B1)} relative to F_{R1(A1)} or in F_{R2(B1)} relative to F_{R2(A1)}. However, if the distal tip were in an appendage or trabeculae—where the entire distal tip were surrounded by tissue, including the outer side antenna/sensor electrode, F_{R1} and F_{R2} for each antenna/sensor electrode (including F_{R1(B1)} and F_{R2(B2)}) would increase. Such a condition for a focal ablation catheter, where contact is not expected on all antenna/sensor electrodes, could indicate an unexpectedly high area of electrode-tissue contact, which could present a risk of steam-pop.

As described above, during ablation, there is a significant change in dielectric properties of tissue (e.g., due to protein denaturation and dehydration with RFA; decrease in cell membrane capacitance with PFA, IRE or PEF; reduced ion mobility in cryoablation, etc.). Each of these mechanisms affect the extent of change and rate of change of HFEPs-particularly in some implementations, changes in F_{R1} and F_{R2}; thus, by monitoring changes in F_{R1} and F_{R2}, one may confirm and assess ablation energy deposition in target tissue and assess and monitor lesion formation.

In some implementations in which RFA ablation is employed (depending on ablation power, which controls speed of ablation and thus speed of change of dielectric properties of the target tissue), F_{R1} may drop for all antenna/sensor electrodes (e.g., as temperature increases, ion mobility increases, and tissue conductivity increases) by about 20-100 MHz (see, for example, F_{R1(C3)} in FIG. 7C-3). F_{R2} may increase gradually for the antenna/sensor electrodes that are in contact with tissue (e.g., F_{R2(C2)} and F_{R2(C3)} in FIGS. 7C-2 and 7C-3); while F_{R2} may not change for antenna/sensor electrodes that are not in contact with tissue (see, for example, F_{R2(C1)} in FIG. 7C-1).

In some implementations, as ablation progresses, intra and extracellular water may decrease, ion mobility may increase, causing F_{R1} to increase and F_{R2} to increase even more. Moreover, for the sensors contacting tissue, F_{R1} may cross the baseline in blood when microbubble formation begins (at about 650 MHz, in some implementations)—which may be a first indication that ablation power should be titrated. In some implementations, a change in F_{R2} of 100-120 MHz may be proportional to about 3.5 to 4.0 mm of lesion depth. If F_{R1} increases to 800 MHZ, in some implementations, the probability of steam pop may increase significantly—and an F_{R1} increase to this level may be another indication to titrate power to prevent adverse events.

The extent of shift in F_{R1} and F_{R2} will depend on the antenna/sensor electrode design, rate of RF power application (for wattages over 50 W, these responses may be different due to higher speed of ablation). To characterize tissue type in contact with an electrode, a library of tissue type and extent and orientation of contact may be made for each antenna/sensor electrode design.

Typically, blood has the highest conductivity and hence the lowest F_{R1} and F_{R2}; healthy myocardium typically has higher a F_{R1} and F_{R2} than blood (about 40 MHz and about 70 MHz, respectively, in some implementations); and ablated tissue may have an F_{R1} about ˜40 MHz higher and an F_{R2} about ˜70 MHz higher than healthy tissue. Fat may have an F_{R1} and F_{R2} which is about 100 MHz and about 300 MHz higher, respectively, than healthy tissue. Scar tissue may have F_{R1} and F_{R2} values between the ablated and fatty myocardium, depending on the extent of fat in the scar tissue.

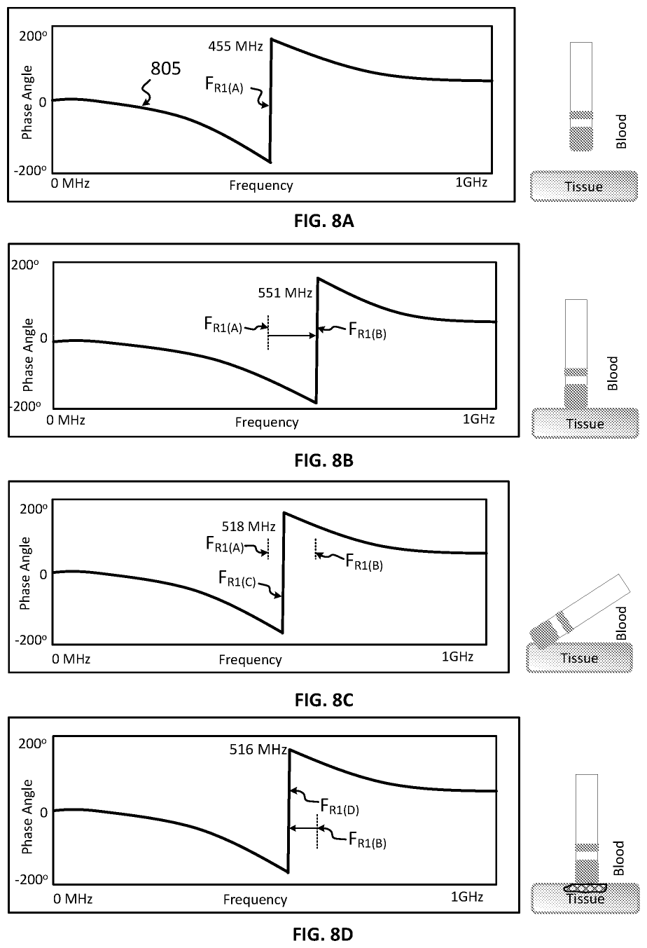

FIGS. 8A-D depict HFEPs in an ablation procedure that employs a dual-electrode antenna/sensor catheter (e.g., as shown in FIGS. 1B and 2A). As with other implementations, phase angle 805 can be monitored, and resonant frequency F_{R1} (phase-reversal frequency) noted.

FIG. 8A depicts a baseline condition, in which the catheter is disposed in blood only. FIG. 8B depicts a condition in the catheter contacts tissue; in this configuration, F_{R1(B)} increases relative to the baseline F_{R1(A)}, based on the orientation and extent of electrode-tissue contact-facilitating use of HFEPs to assess and confirm contact as described elsewhere herein. (To illustrate the impact of reduced tissue contact, FIG. 8C depicts a decrease in F_{R1(C)}, relative to F_{R1(B)} (but still an increase, relative to F_{R1(A)}) when tissue contact is reduced (e.g., as a result of oblique contact)).

During either PFA or RFA ablation, there may be a distinct change to HFEPs. For example, as ablation in initiated, F_{R1(D)} drops, relative to F_{R1(C)}, indicating energy deposition in the tissue and lesion formation (see FIG. 8D). In some implementations, particularly with spiral antenna/sensor electrodes in which PFA ablation is employed, there is a drop in cell membrane capacitance of ablated tissue, due to cell wall disruption. This may be confirmed by an increase in F_{R1} and F_{R2} relative to non-ablated tissue. During PFA, to confirm the applied voltage is resulting in appropriate lesion formation, F_{R1} and F_{R2} can be monitored. As PFA lesion formation progresses, F_{R1} and F_{R2} increase relative to healthy, non-ablated tissue-proportional to irreversible cell membrane injury. In some implementations, a change of about 50 MHz for F_{R1} and about 100 MHz for F_{R2} is indicative of irreversible PFA lesion formation. However, these changes are antenna/sensor electrode design specific and would need to be benchmarked for each design.

To intraoperatively monitor and assess lesion formation during PFA, electrode tissue contact can be confirmed by increases in F_{R1} and F_{R2} from baseline in blood (for spiral antenna/sensor electrodes, F_{R1} and F_{R2} on healthy myocardium may be higher by about 50 MHz and 90 MHz than in blood). During PFA voltage application, as lesion formation progresses, F_{R1} and F_{R2} increase by about 50 MHz and about 200 MHz, respectively. When the F_{R2} increase reaches a critical rise of about 250 MHz, voltage application may be stopped, as a permanent increase of F_{R2} of about 100 MHz post voltage pulse application typically predicts an irreversible lesion.

In case of microbubbles, the Logarithm of the Reflection Coefficient and phase angle traces may get irregular and flicker. If at any point during the procedure, tissue contact is lost, F_{R1} may drop below F_{R1} (A); thus, if F_{R1} does drop below F_{R1} (A), loss of tissue contact can be inferred, and the catheter position can be adjusted to reestablish tissue contact.

### Sensing Force of Contact and Area of Contact

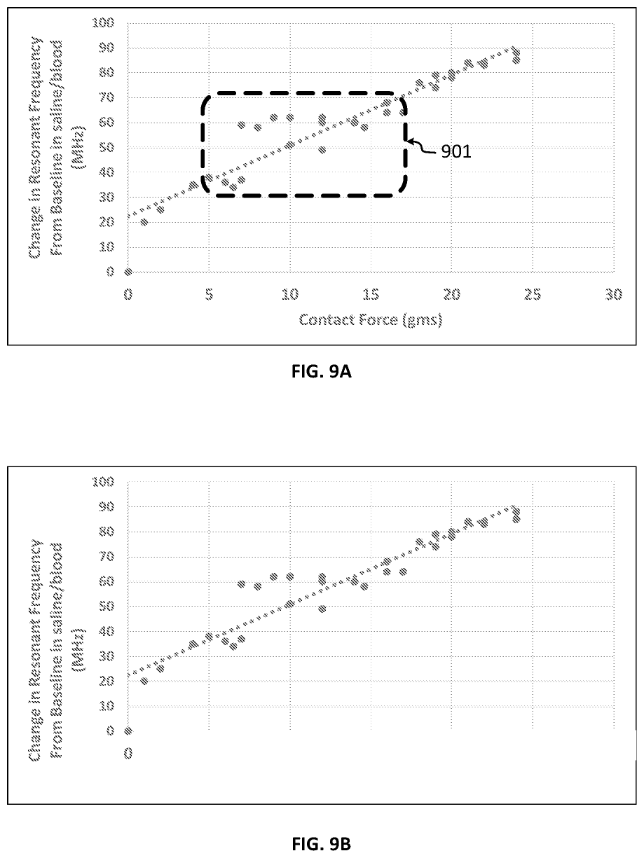

As FIGS. 8B and 8C illustrate, extent of contact (and force of contact) can be inferred from changes in F_{R1}, relative to a baseline with blood-only contact. Turning to FIG. 9A, a plot, is provided showing a correlation, in one implementation, between contact force in grams (x-axis) and change in resonant frequency (e.g., F_{R1}) (y-axis) from baseline (blood-only contact) to tissue contact. A desirable range 901 is highlighted-indicating that, for the implementation shown, in order to have between about 5 grams and about 17 grams of contact force, a change in resonant frequency of about 30 MHz to about 70 MHz is desirable. The precise correlation between grams of force and change in resonant frequency may be dependent on the specific antenna/sensor electrode design and type of tissue and/or bodily fluid; but in many implementations, the correlation can be calibrated. This correlation may enable a caregiver to modify a procedure to avoid specific risks. For example, if a contact force of greater than 20 grams is undesirable in the implementation depicted, a physician may watch for a shift in resonant frequency of more than about 75 MHz; and if that level of shift is identified, the contact force may be reduced.

FIG. 9B illustrates a similar correlation between area of contact of an antenna/sensor electrode (x-axis) and change in resonant frequency (y-axis). This correlation may also be dependent on the specific antenna/sensor electrode design and type of tissue and/or bodily fluid; and it, too, may calibrated. In some implementations, the correlation of FIG. 9A and that of FIG. 9B are independent of each other; in other implementations, the correlations may be related. As shown, FIGS. 9A and 9B may be related; and as the reader will appreciate, the desirable ranges of area of contact 902, force of contact 901 and change in resonant frequency, as shown, do not perfectly align. That is, FIG. 9B depicts a desirable contact area 902 as corresponding to a shift of about 50-95 MHz, relative to a baseline; however, the desirable contact force 901, from FIG. 9A, is between about 30 MHz and 70 MHz. Accordingly, in such an implementation, it may be desirable to target a shift in resonance frequency of between about 50 MHz and about 70 MHz—and to manipulate the distal tip of a corresponding catheter until this level of contact force and contact area are inferred.

In some implementations, it may be desirable to include a dedicated force sensor, rather than inferring contact force from change in other HFEPs associated with antenna/sensor electrodes that may also be used to perform ablation. FIG. 10A illustrates one exemplary force sensor 1001 that may be integrated into an ablation catheter. In some implementations, the force sensor 1001 could be a fiberoptic force transducer; in other implementations, as shown, the force sensor 1001 could include electromagnetic resonator 1003 (e.g., a solenoid coil with at least one turn, the ends of which may be coupled to a coaxial cable-such that the opposite end could be coupled to a VNA or other network analyzer that could detect a change in the resonance of the coil as force is applied). In some such implementations, the resonant frequency or logarithm of the reflection coefficient could be used to infer applied force. FIG. 10B illustrates the sensor 1001 of FIG. 10A integrated into the distal tip of an ablation catheter, in one implementation. In implementations that include a dedicated force sensor, force sensor readings may be used in conjunction with other available data to characterize tissue and assess lesion formation.

### Ablation at Multiple Points Simultaneously

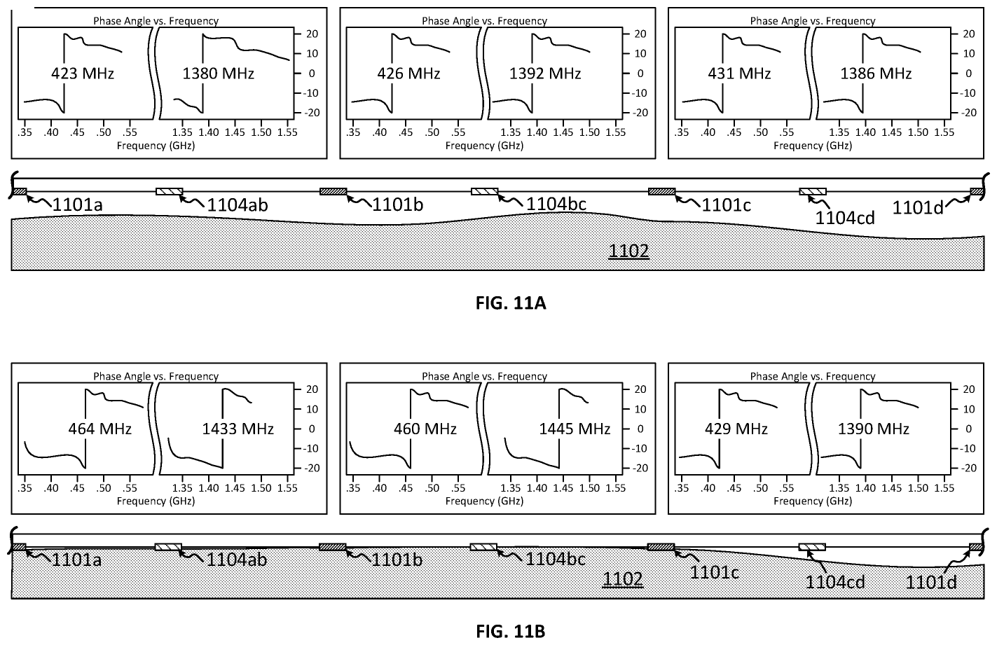

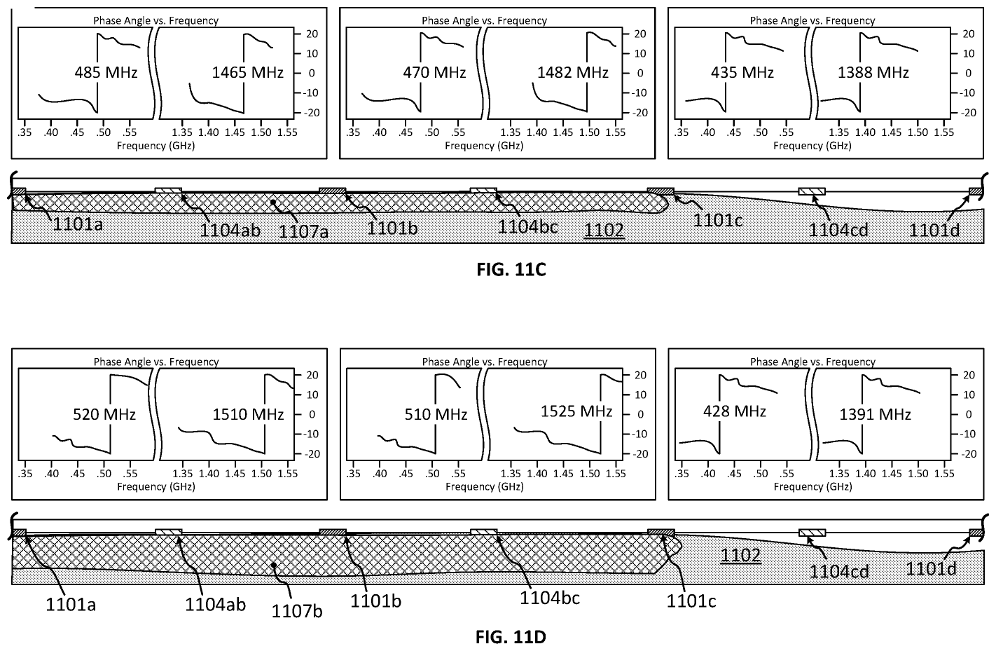

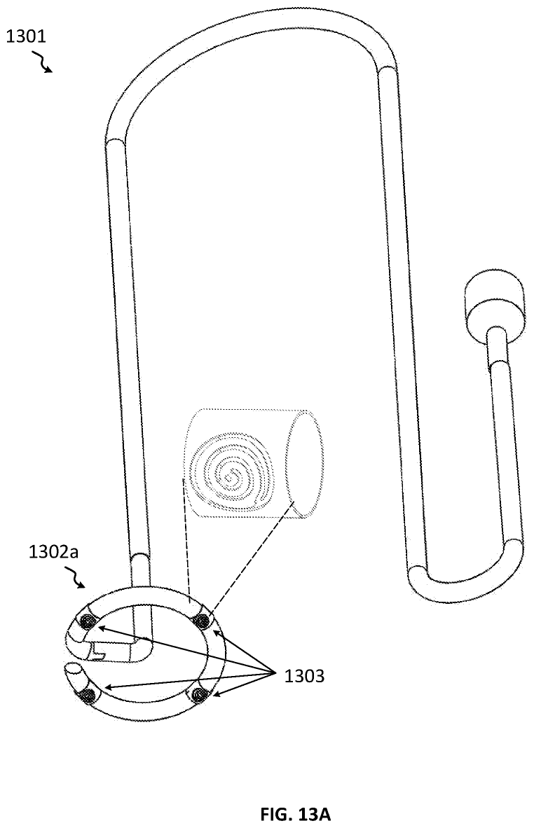

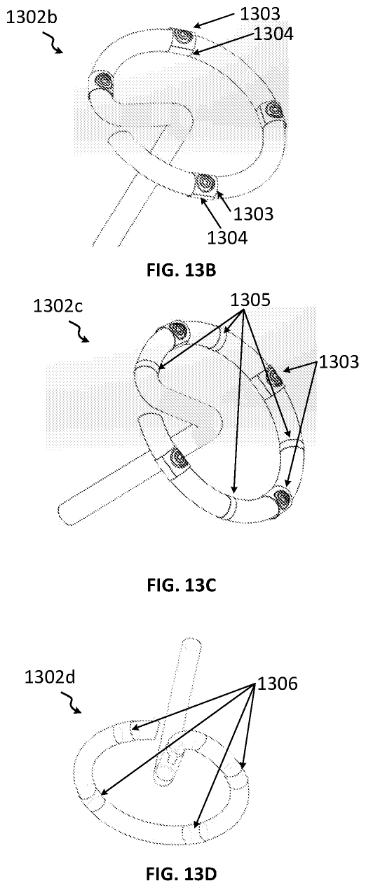

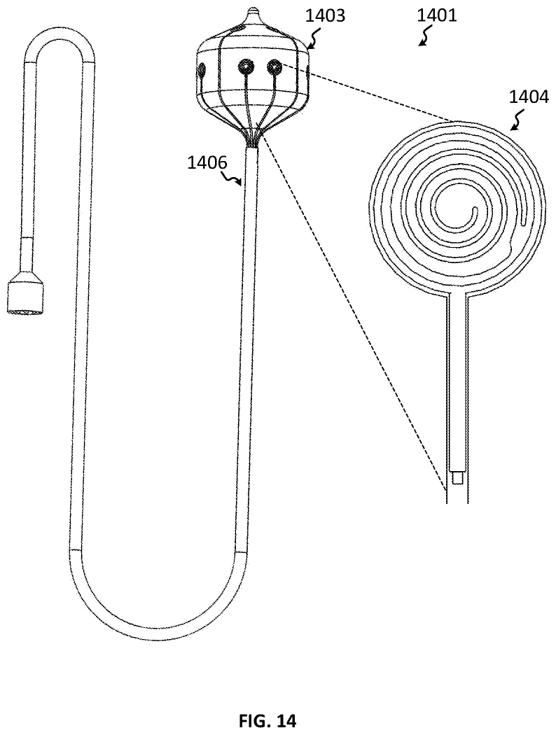

FIGS. 11A-11D depict an implementation in which an PEF/PF ablation procedure may proceed on tissue 1102 at multiple points simultaneously. For example, such an ablation procedure may be performed with a catheter having a distal end like that shown in FIG. 13C-having separate ablation ring electrodes 1305 and spiral antenna/sensor electrodes 1303 between pairs of ablation electrodes. As shown, four ablation electrodes are provided—1101a, 1101b, 1101c and 1101d (where ablation energy is applied across pairs of electrodes); and three antenna/sensor electrodes—1104ab, 1104bc and 1104cd—are provided between pairs of ablation electrodes. HFEPs are monitored from the antenna/sensor electrodes 1104ab, 1104bc and 1104cd, as in other implementations; and as shown, baseline values for F_{R1} and F_{R2} with blood-only contact are obtained at each antenna/sensor 1104ab, 1104bc and 1104cd.

FIG. 11B illustrates contact between tissue 1102 and ablation electrodes 1101a, 1101b, 1101c, and interposed antenna/sensor electrodes 1104ab and 1104c; as shown, there is no tissue contact with sensor 1104cd. As with other implementations, F_{R1} and F_{R2} values are shown to reflect the status of tissue contact—for those antenna/sensor electrodes 1104ab and 1104bc that are in contact with tissue 1102, F_{R1} values are shown as increased relative to the baseline by about 40 MHz; and F_{R2} values are shown as increased relative to the baseline by about 50 MHz. In contrast, the F_{R1} and F_{R2} values associated with the antenna/sensor electrode 1104cd that is not in contact with tissue 1102 is relatively unchanged.

In implementations like this, ablation energy may be selectively delivered only to those pairs of ablation electrodes whose corresponding antenna/sensor electrodes indicate tissue contact (e.g., a separate antenna/sensor electrode, as shown; or, in other implementations, where sensing occurs at the ablation electrode, the ablation electrode itself); and those ablation electrodes that are determined to not be in contact with tissue may not receive ablation energy.

FIG. 11C depicts a procedure in which ablation energy has been delivered across the ablation electrodes 1101a and 1101b, and across ablation electrodes 1101b and 1101c. As shown, a shallow lesion 1107a has started forming, and F_{R1} and F_{R2} values reflect this initial lesion formation. That is, F_{R1} values are shown as having decreased (relative to tissue contact prior to ablation, as depicted in FIG. 14B) by about 15-30 MHz; and F_{R2} values are shown as having increased by about 30-50 MHz.

FIG. 11D depicts continuation of the ablation procedure. A deeper lesion 1107b is shown as having been formed, and F_{R1} values and F_{R2} values have continued shifting. As shown, F_{R1} values have increased about another 25 MHz (for a total increase relative to tissue contact prior to ablation of about 40-50 MHZ); and F_{R2} values have increased by about another 45 MHz (for a total increase relative to tissue contact prior to ablation of about 75-100 MHz).

In the manner just described and illustrated with respect to FIGS. 11A-11D, multiple points of target tissue may be selectively ablated simultaneously; while ablation energy may be blocked from being delivered from ablation electrodes that are determined to not be in contact with target tissue. Such an approach—where ablation energy is delivered to multiple points simultaneously—can, in some implementations, reduce procedure time. For example, with a circumferential distal tip with a plurality of ablation electrodes and corresponding antenna/sensor electrodes, it may be possible to ablate a significant portion of the circumference of a pulmonary vein ostium (or at least many points along the circumference—such that the ablation catheter may be rotated slightly one or more times to conclude an ablation procedure on that ostium-without requiring point-by-point ablation around the circumference).

Numerous variations are possible. For example, only three antenna/sensor electrodes are shown in FIGS. 11A-11D; but in some implementations, there may be eight such antenna/sensor electrodes and eight corresponding ablation electrodes. Other implementations may have four of each ablation electrodes and antenna/sensor electrodes; other implementations may have dual-purpose electrodes, in which ablation energy is delivered from the same electrodes that provide sensing capabilities.

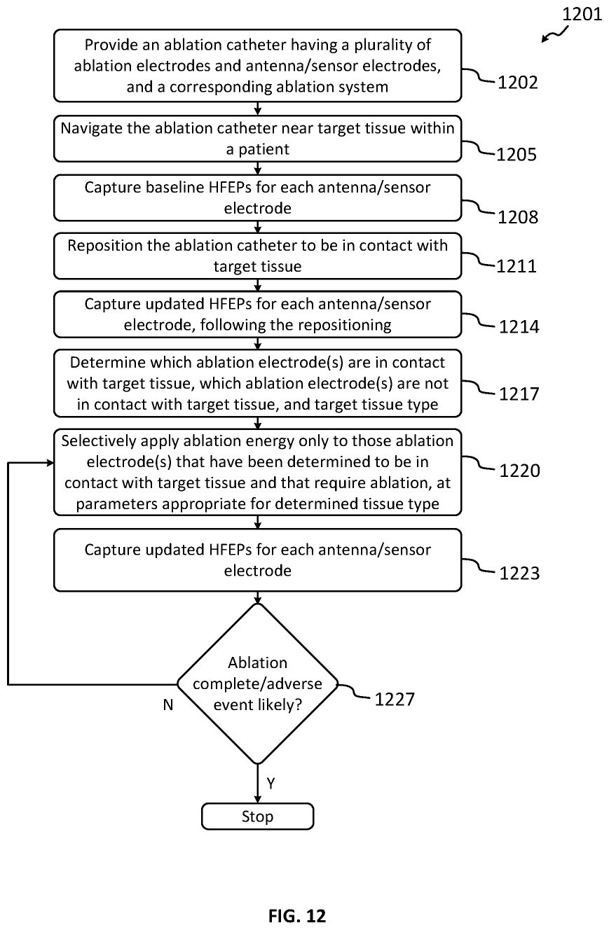

FIG. 12 illustrates an exemplary method 1201 for ablating target tissue. As shown, the method includes providing (1202) an ablation catheter having a plurality of ablation electrodes and antenna/sensor electrodes, and a corresponding ablation system. In some implementations, the ablation catheter has a distal ablation tip such as one shown in FIG. 13B or 13C; and the ablation system may have a configuration like the system 401 shown in FIG. 4—with a VNA or VNA channel coupled to each antenna/sensor electrode on the ablation catheter. Various forms of ablation that are described herein may be provided, and the ablation energy may be delivered by dedicated ablation electrodes or by combination ablation and antenna/sensor electrodes.

The method 1201 includes navigating (1205) the ablation catheter near target tissue within a patient. For example, with reference to FIG. 4, a physician may navigate (1205) the catheter 405 into a patient's left atrium in order to perform a pulmonary vein isolation procedure.

The method 1201 includes capturing (1208) baseline HFEPs for each antenna/sensor electrode. For example, with reference to FIG. 11A, baseline phase-reversal frequencies F_{R1} and F_{R2} could be captured for each antenna/sensor electrode 1104ab, 1104bc and 1104cd.

The method 1201 includes repositioning (1211) the ablation catheter to be in contact with target tissue. For example, with reference to FIG. 4, a physician may reposition (1211) the catheter 405 against a specific pulmonary vein antrum of the patient 402—using, for example, imaging equipment 404 and/or the cardiac mapping and navigation system 407 within the ablation system 401.

The method 1201 includes capturing (1214) updated HFEPs for each antenna/sensor electrode, following the repositioning (1211). For example, with reference to FIG. 11B, updated phase-reversal frequencies F_{R1} and F_{R2} may be captured for each antenna/sensor electrode 1104ab, 1104bc and 1104cd.

The method 1201 includes determining (1217), from the updated HFEPs, which antenna/sensor electrode(s) (and/or corresponding ablation electrode(s)) are in contact with target tissue, which antenna/sensor electrode(s) (and/or corresponding ablation electrode(s)) are not in contact with target tissue, and the tissue type (e.g., healthy tissue, scar tissue, partially ablated tissue). For example, with reference to FIG. 4 and FIG. 11B, the ablation system 401 may determine that ablation electrodes 1101a, 1101b and 1101c are in contact with target tissue 1102, but ablation electrode 1101d is not in contact with target tissue 1102. More specifically, the system 401 may determine that F_{R1} and F_{R2} parameters have increased by specific threshold amounts for specific antenna/sensor electrodes and thereby determine tissue contact. The ablation system 401 may further determine that the tissue is healthy (e.g., not scarred or already ablated) tissue.

The method 1201 includes selectively applying (1220) ablation energy only to those ablation electrode(s) that have been determined to be in contact with target tissue, at parameters appropriate for the determined tissue type. For example, with reference to FIGS. 4 and 11C, the system 401 may apply ablation energy across electrodes 1101a and 1101b and across electrodes 1101b and 1101c. The level of energy may be adjusted based on the tissue type or based on other potentially likely adverse events. For example, if the tissue type is determined to be scar tissue, ablation energy may be increased relative to tissue type that is determined to be healthy. Similarly, if already ablated tissue is detected, ablation energy may be adjusted based on a determined lesion depth (e.g., in some implementations, ablation energy may be lowered as a desirable depth is approached, to avoid ablating too deep). For thin tissue (e.g., tissue on a posterior wall of the heart), the ablation parameters and duration may be adjusted.

The method 1201 includes capturing (1223) ablation HFEPs for each antenna/sensor electrode. For example, with reference to FIGS. 4 and 11C, the ablation system 401 may capture updated phase-reversal frequencies F_{R1} and F_{R2} may be captured for each antenna/sensor electrode 1104ab, 1104bc and 1104cd.

The method 1201 includes determining (1227) whether the ablation procedure is complete and/or whether an adverse event is likely. For example, with reference to FIGS. 4 and 11D, the ablation system 401 may determine that F_{R1} and F_{R2} parameters for ablation electrodes to which was ablation energy was delivered increased by a sufficient threshold (e.g., 50-100 MHZ, or 75-100 MHz, or 75-85 MHZ, in various implementations), relative to like parameters in blood or like parameters at pre-ablation tissue contact) that ablation is completed (e.g., that the lesion depth 1107b meets clinical objectives in terms of depth and extend). If the ablation system 401 determines (1227) that the ablation procedure is complete, ablation may be concluded; alternatively, step 1220 of selectively applying ablation may be continued. As another example, if an unexpected level of noise in F_{R1} and F_{R2} parameters is detected, the ablation system 401 may determine that microbubbles have started forming, and ablation may be stopped. As another example, if a drop in F_{R1} or F_{R2} below baseline values is detected, the ablation system 401 may determine that tissue contact has been lost, and ablation may be stopped, or stopped with respect to the corresponding ablation electrode for which lost contact was determined. In some implementations, the ablation system 401 may provide an alarm or status indication to enable a caregiver to review and reposition the ablation catheter as necessary. If it is determined that an adverse event is likely, ablation may be stopped, or ablation parameters may be adjusted and the ablation continued (e.g., at step 1220).

In some implementations, the method 1201 may include additional steps. For example, a force sensor may be employed to provide additional positioning information (e.g., contact force); and based on the positioning information, the catheter may be repositioned. In implementations in which additional ablation is required at a slightly different spot (e.g., if a circumferential catheter needs to be rotated to ablate additional antrum regions around an ostium of a particular pulmonary vein, or if another pulmonary vein must be treated), the catheter may be repositioned, and steps 1205 to 1227 may be repeated.

### Additional Exemplary Catheter Designs

Additional exemplary catheter designs are now described. FIG. 13A shows a multi-electrode circular catheter 1301 with its electrodes disposed at a distal end 1302a and configured as spiral antenna/sensor electrodes 1303. Each spiral antenna/sensor 1303 may be connected via a high frequency transmission line, such as a coaxial cable, such that the core of the coaxial cable is connected to the spiral and the shield to an adjacent ground electrode. With reference to FIG. 4, each antenna/sensor electrode 1303 may be connected to a VNA or VNA channel (e.g., 419a, 419b, 419c, etc.) and an ablation generator (e.g., 413a, 413b, 413c, etc.) through a filter circuit (e.g., 422a, 422b, 422c, etc.). Such a configuration may enable simultaneously delivery of ablation signals (e.g. high voltage PEF/IRE pulse trains, RF signals, etc.) and high-frequency assessment signals for lesion assessment with the same spiral antenna-sensor electrodes 1303.

FIG. 13B illustrates a variation of the distal end 1302b of FIG. 13A. As shown, spiral antenna/sensor electrodes 1303 may be disposed adjacent ground electrodes 1304. FIG. 13C illustrates another variation of the distal end 1302c in which ring electrodes 1305 may be disposed between antenna/sensor electrodes 1303. FIG. 13D illustrates yet another variation of the distal end 1302c in which spiral antenna/sensor electrodes are replaced solely with ring electrodes 1306, where an electrode pair forms a dual electrode antenna.

In the various implementations illustrated and described in FIGS. 13A-D and throughout this disclosure, PEF or IRE or high voltage ablation pulses may be delivered via antenna/sensor electrodes in multiple ways. For example, they may be delivered in a biphasic-bipolar manner between two electrodes on a catheter, through an antenna/sensor electrode and a skin electrode (e.g., electrode 428 in FIG. 4), between an antenna/sensor electrode and an adjacent ground electrode, in biphasic-monopolar configurations, etc. In implementations in which both spiral antenna/sensor electrodes and ring electrodes are present, the ring electrodes may be used to apply and deliver high voltage PEF ablation pulses, and the spiral antenna/sensor electrodes may be used to assess procedure parameters and confirm durable contagious lesion formation.

Other catheter variations are possible. For example, FIG. 14 illustrates a multi-electrode balloon catheter 1401 where multiple spiral antenna/sensors electrodes (e.g., electrode 1404) are incorporated on the outer surface of the balloon portion 1403 (which may be rigid, semi-compliant or compliant). The electrodes 1404 may be printed directly on the surface of the balloon 1403 or incorporated on the balloon 1403 by printed flex circuits. The flex circuits may have spiral antenna/sensors electrodes 1404, coaxial antenna sensors or any other type of antenna configured on the flex board, which may have a transmission line (strip line transmission cable, etc.) configured to route the circuit over the balloon 1403 and connect to another coaxial cable (not shown) inside the catheter body 1406) or a high frequency transmission line that runs along the length of the catheter.

During clinical use, the balloon catheter 1401 may be advanced to the cardiac anatomy of interest (e.g. pulmonary vein ostia, via the femoral vein) using electromagnetic catheter navigation (e.g. EnSite Precision), and the balloon may be inflated such that a maximum number of electrodes 1404 are in contact with the cardiac anatomy of interest (e.g., the catheter 1401 may be positioned such that the pulmonary vein antrum anatomy of interest to be ablated is in contact with the antenna-sensor electrodes 1404 on the balloon 1403, and PEF voltages may be applied to create a contiguous lesion). As with other implementations, lesion formation may be assessed and confirmed by shifts in HFEPs (e.g., shifts in F_{R1} and F_{R2}) for each individual antenna/sensor electrode 1404. After an initial ablation procedure, the balloon 1401 may be rotated to assess gaps in lesions, and additional ablation may be delivered to fill any such gaps.

Various PEF application schemes may be used—for example, voltage pulses may be applied between two or more adjacent electrodes, or by the same electrode using a spiral antenna/sensor electrode as the positive and ground plane as the negative. Similarly, bipolar monophasic, monopolar-biphasic, and other pulse forms of PEF, PFA or IRE pulses may be applied using the grounding pad (e.g., ground pad 428 in FIG. 4) as the other electrode.

The balloon catheter 1401 may also be used to monitor cryoablation procedures. That is, a cryo fluid may be delivered to the balloon to create lesions; and the electrodes 1404 may be used as described herein to assess and monitor lesion formation.

The balloon catheter 1401 may also be used with RF ablation—in which case, the balloon catheter 1401 may be modified with microholes for irrigation. In such implementations, the balloon 1401 could be inflated with a suitable fluid (e.g., saline), the pressure may be adjusted to maximize electrode-tissue contact, and RF ablation signals may be delivered to the tissue via spiral antenna/sensor electrodes 1404—between the electrodes and/or between electrodes and the ground pad. Power to each antenna/sensor electrode 1404 may be regulated to ensure safe and efficacious ablation. As with other implementations, location of the antenna/sensor electrodes within target anatomy may be tracked and voltage maps may be acquired using IEGM measurement systems and cardiac mapping and navigation systems.

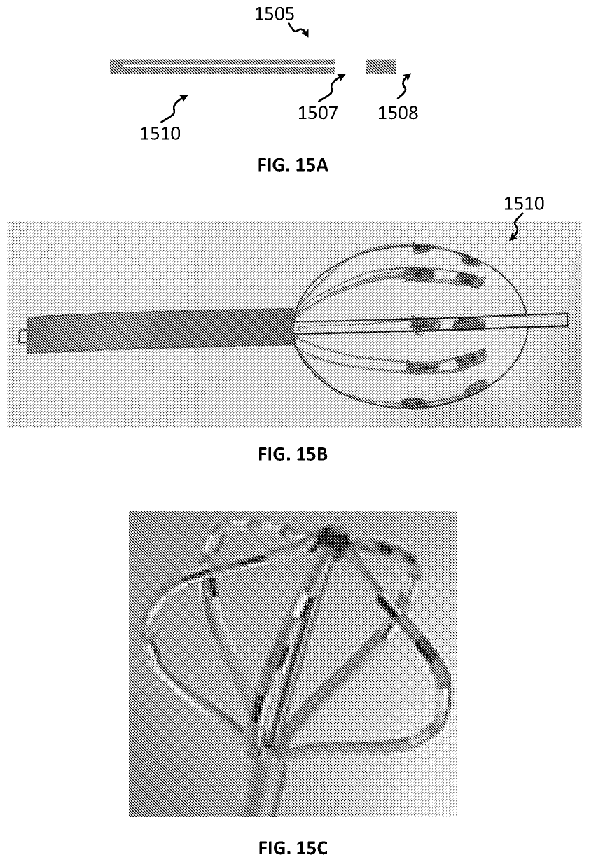

FIG. 15A illustrates a dipole antenna design 1505 that may be used in place of the spiral antenna/sensor electrodes of other implementations. As shown, the design 1505 includes two electrodes, 1507 and 1508—on of which may be coupled to the inner conductor of a coaxial cable 1510, and the other of which may be coupled to the shield of the coaxial cable 1510.

In some implementations, as shown in FIG. 15B, such a design 1505 may be incorporated on a balloon. 1515. The surface of the balloon 151 may have more than one antenna element, each with two electrodes, which may be connected to dedicated coaxial cables for assessment and delivering the high voltage ablation pulses. During clinical use of such a balloon 1515, the balloon could be advanced to the anatomy of interest, electrode-tissue contact could be confirmed as described herein by monitoring HFEPs, tissue type could be assessed, and ablation voltage and pulse parameters could be adjusted and delivered by adjusting the voltage delivery pattern among the electrodes to create a desired e-field pattern. Such a dipole antenna design could also be incorporated on a basket catheter with multiple splines and similarly used for circular ablations, as shown in FIG. 15C.

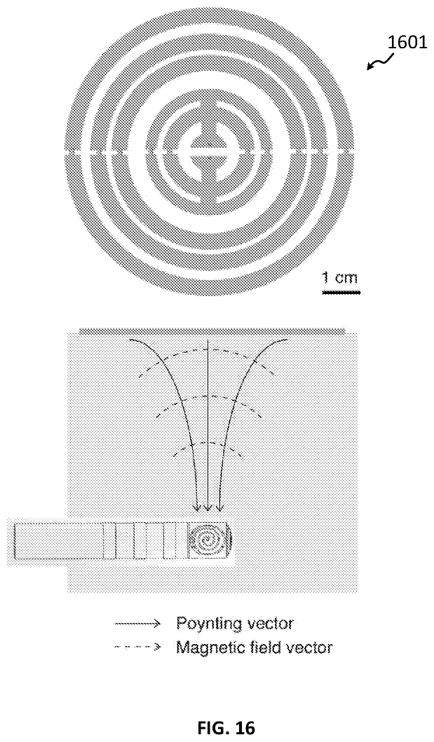

In some implementations, the high-frequency sensing and assessment of lesions may be limited. That is, the depth of penetration of the signals in lossy tissue media may be only 2-5 mm. To improve performance in assessing deeper lesions (e.g. for tumors, or ventricle ablations), a midfield antenna 1601, shown in FIG. 16, may be placed outside the body (e.g., on the surface of a patient's skin, in line with an ablation catheter or an ablation needle inside the body). Such an antenna 1601 can couple to antenna/sensor electrodes in such a way that HFEPs may be changed relative to what they would be in the absence of the antenna 1601. With these changes to HFEPs (specifically, for example, changes to amplitudes and phases of transmission signals between ablation electrodes and the external midfield antenna 1601), deeper lesions depths may be assessed. Such methodology may also facilitate greater precision in tracking of the location of a catheter coil internally in the body (e.g., by monitoring changes to HFEPs within the field of the antenna 1601).

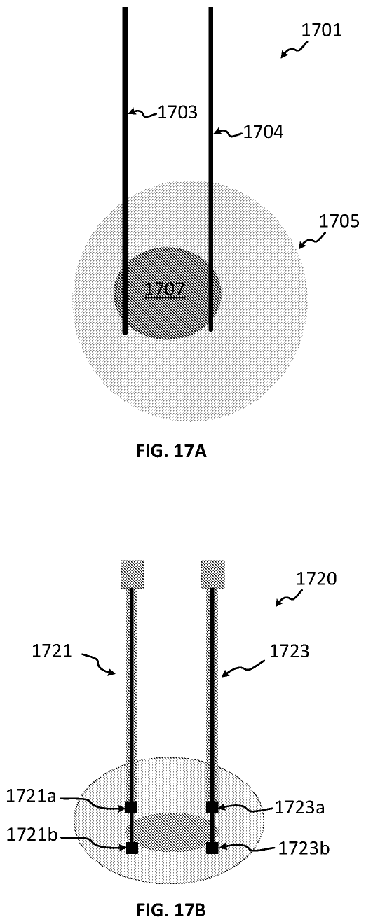

FIG. 17A illustrates an exemplary configuration 1701 for tumor ablation. In the configuration 1701, at least two needles (e.g., needle 1703 and needle 1704) may be disposed in tissue 1705 to be ablated. The needles 1703 and 1704 may be configured as parallel conductor antenna and driven with appropriate ablation and high frequency sensing signals described herein. For example, needle 1703 could serve as a positive electrode for PFA ablation, and needle 1704 could serve as the ground, and at the same time serve as positive and ground of the antenna. HFEPs could then be monitored to assess formation of a lesion 1707 between the needle electrodes 1703 and 1704.

Because many tumor cells have a distinct high frequency signature, complete ablation of a tumor mass may be detected by monitoring the HFEPs of the needle configuration 1701. In some implementations, multiple pairs of needles may be employed—where one or more needle-antenna pairs can be used for PFA and one or more needle-antenna pairs can be used for HFDS assessment. HFEPs may be measured in reflection mode and transmission mode to enable accurate assessment of tumor ablation.

FIG. 17B illustrates another implementation in which needle electrodes 1721 and 1723 are configured as modified dual-electrode dipole antenna (with electrodes 1721a and 1723a coupled to a shields of coaxial cables, and electrodes 1721b and 1723b coupled to inner conductors of the coaxial cables, respectively). In such implementations, high-frequency reflection and transmission impedance properties of the two or more needles, while delivering the ablation energies (e.g., RFA or PFA, or Cryo) can be monitored and analyzed to infer the extent of ablation, including complete irreversible ablation.

Several implementations have been described with reference to exemplary aspects, but it will be understood by those skilled in the art that various changes may be made and equivalents may be substituted without departing from the contemplated scope. For example, method steps may be reordered, and certain steps may be omitted while other steps may be added. Various modalities of ablation are described which may be employed, and in some implementations, multiple modalities of ablation may be provided by a single catheter or system. Various antenna/sensor electrodes designs are described, and multiple antenna/sensor electrode designs may be employed on a single catheter or within a single system. Specific design elements of the antenna/sensor electrodes may be made; for example, dimensions of individual components could be increased or decreased, metal thicknesses and types could be varied, greater or fewer turns may be provided in spiral designs, greater or fewer antenna/sensor electrodes may be provided on a distal end. Heart procedures are described, but the systems and methods described herein could be applied to tissue other than heart tissue. Voltages, frequencies and frequency ranges may be varied relative to this disclosure. Phase-reversal parameters F_{R1} and F_{R2} are described, but other HFEPs may be relevant, including other phase-reversal parameters (e.g., F_{R3} or F_{R4}—i.e., higher-frequency resonant frequencies, e.g., in the 5-10 GHz range, in the 10-15 GHz range, or at even higher frequencies).

Many other variations are possible, and modifications may be made to adapt a particular situation or material to the teachings provided herein without departing from the essential scope thereof. Therefore, it is intended that the scope include all aspects falling within the scope of the appended claims.

What is claimed is:

1. A method comprising:
providing an ablation system having (i) an ablation catheter having a plurality of antenna/sensor electrodes at a distal end; (ii) a plurality of ablation generators that are each configured to generate ablation signals; (iii) a plurality of vector network analyzers (VNAs); (iv) a filtering system having a plurality of channels; and (v) a transmission system; wherein the transmission system individually couples each of the plurality of antenna/sensor electrodes to one discrete ablation generator in the plurality of ablation generators and one discrete VNA in the plurality of VNAs, through one discrete channel in the plurality of channels in the filtering system; wherein each VNA in the plurality of VNAs is configured to (A) transmit sensing signals across a spectrum of frequencies; (B) measure transmit power for the transmitted signals and measure received power for reflected-back signals; and (C) from the measured transmit power and measured received power, calculate high-frequency electrical parameters (HFEPs); and wherein each channel in the filtering system prevents ablation signals from interfering with sensing signals;
navigating the ablation catheter to a target treatment region within a patient;
with the plurality of VNAs, capturing baseline HFEPs for each antenna/sensor electrode;
positioning the ablation catheter to be in at least partial contact with target tissue;
with the plurality of VNAs, capturing updated HFEPs for each antenna/sensor electrode;
identifying, from the updated HFEPs, a first subset of antenna/sensor electrodes that are in contact with the target tissue and a second subset of antenna/sensor electrodes that are not in contact with the target tissue; and
with a subset of the ablation generators, selectively providing ablation signals to the first subset of antenna/sensor electrodes but not to the second subset of antenna/sensor electrodes.

2. The method of claim 1, wherein the HFEPs comprise a first phase-reversal frequency parameter, F_{R1}, and a second phase-reversal frequency parameter, F_{R2}, for each antenna/sensor electrode.

3. The method of claim 2, wherein identifying the first subset of antenna/sensor electrodes that are in contact with the target tissue comprises determining that F_{R1} and F_{R2} parameters in the updated HFEPs are each greater than corresponding F_{R1} and F_{R2} parameters in the baseline HFPEs by a threshold frequency.

4. The method of claim 3, wherein the threshold frequency is 30 MHz.

5. The method of claim 3, wherein the threshold frequency is between 25 MHz and 50 MHz.

6. A method of claim 2, further comprising:
with the plurality of VNAs, capturing ablation-progress HFEPs;
determining from the ablation-progress HFEPs whether ablation parameters (A) meet clinical objectives, (B) indicate a likelihood of adverse events, or (C) do not yet meet clinical objectives; and
if ablation parameters are determined to meet clinical objectives, stopping the selective application of ablation energy; if the ablation parameters are determined to indicate a likelihood of adverse events either adjusting the selective application of ablation energy or stopping the selective application of ablation energy; and if the ablation parameters are determined to not yet meet clinical objectives, continuing the selective application of ablation energy.

7. The method of claim 6, wherein determining that ablation parameters meet clinical objectives comprises determining that F_{R1} parameters in the ablation-progress HFEPs are greater than corresponding F_{R1} parameters in the updated HFPEs by a first threshold frequency, and F_{R2} parameters in the ablation-progress HFEPs are greater than corresponding F_{R2} parameters in the updated HFPEs by a second threshold frequency.

8. The method of claim 7, wherein the first threshold frequency is about 30 MHz, and the second threshold frequency is about 20 MHz.

9. The method of claim 6, wherein determining that ablation parameters meet clinical objectives comprises determining that F_{R1} parameters in the ablation-progress HFEPs are greater than corresponding F_{R1} parameters in the baseline HFPEs by a first threshold frequency, and F_{R2} parameters in the ablation-progress HFEPs are greater than corresponding F_{R2} parameters in the updated HFPEs by a second threshold frequency.

10. The method of claim 9, wherein the first threshold frequency is about 30 MHz, and the second threshold frequency is about 50 MHz.

11. The method of claim 1, wherein the ablation system further comprises (vi) a cardiac mapping and navigation system; (vii) a controller; and (viii) and a switch that selectively couples or decouples the cardiac mapping and navigation and the transmission system; wherein the controller causes the switch to decouple the cardiac mapping and navigation system and the transmission system when the subset of ablation generators selectively provides ablation signals to the first subset of antenna/sensor electrodes.

12. The method of claim 11, wherein positioning the ablation catheter to be in at least partial contact with target tissue comprises positioning the ablation catheter based on information received from the cardiac mapping and navigation system.

13. The method of claim 11, wherein positioning the ablation catheter to be in at least partial contact with target tissue comprises positioning the ablation catheter based on information received from imaging equipment that is external to the ablation system.

14. The method of claim 1, wherein the transmission system comprises a plurality of coaxial cables, wherein a discrete coaxial cable couples each antenna/sensor electrode to a discrete channel in the filtering system.

15. The method of claim 1, wherein at least one antenna/sensor electrode in the plurality of antenna/sensor electrodes is configured as a spiral antenna/sensor electrode having at least two turns.

16. The method of claim 1, wherein the ablation signals are radio-frequency ablation (RFA) signals.

17. The method of claim 1, wherein the ablation signals are pulse-field ablation (PFA) signals.

18. The method of claim 17, wherein the PFA signals comprise trains of high-voltage pulses of at least 1 KV, delivered at a field strength of at least 100 V/cm.

19. A method comprising:
providing an ablation system having (i) an ablation catheter having a plurality of ablation electrodes and a plurality of antenna/sensor electrodes at a distal end; (ii) a plurality of ablation generators that are each configured to generate ablation signals; (iii) a plurality of vector network analyzers (VNAs); (iv) a filtering system having a plurality of channels; and (v) a transmission system; wherein the transmission system individually couples each of the plurality of ablation electrodes to one discrete ablation generator in the plurality of ablation generators and each of the plurality of antenna/sensor electrodes to one discrete VNA in the plurality of VNAs, through one discrete channel in the plurality of channels in the filtering system; wherein each VNA in the plurality of VNAs is configured to (A) transmit sensing signals across a spectrum of frequencies; (B) measure transmit power for the transmitted signals and measure received power for reflected-back signals; and (C) from the measured transmit power and measured received power, calculate high-frequency electrical parameters (HFEPs); and wherein each channel in the filtering system prevents ablation signals from interfering with sensing signals;
navigating the ablation catheter to a target treatment region within a patient;
with the plurality of VNAs, capturing baseline HFEPs for each antenna/sensor electrode;
positioning the ablation catheter to be in at least partial contact with target tissue;
with the plurality of VNAs, capturing updated HFEPs for each antenna/sensor electrode;
with the data processor, identifying, from the updated HFEPs, a first subset of ablation electrodes that are determined to be in contact with the target tissue and a second subset of ablation electrodes that are determined to not be in contact with the target tissue; and
with a subset of the ablation generators, selectively providing ablation signals to the first subset of ablation electrodes but not to the second subset of ablation electrodes.

20. The method of claim 19, wherein each antenna/sensor electrode in the plurality of antenna/sensor electrodes is disposed between two ablation electrodes in the plurality of ablation electrodes.
